SEQUENCE LISTING (6 sequences)

SEQ ID NO: 1 - DNA, 3435 bp, Homo sapiens
  CDS (507)..(1487)
acagcaactatgaaataatcgtagtatgagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacgtgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggtattccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcatgccattacagctttctagatgctaatacatgtgactatcgtccaatttgctttcttttgtagtgacattaaatttggctataaaagatggactacatgtgatactcctatggacgttaattgaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaacaagtgctttttatcttgaaattcaaccaacggaaagaatatgcatagaataatgcattctatgtagccatgtcactgtgaataacgatttcttgcatatttagccattttgattcctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatttataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaagattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcacccatcagttatttgtgagggtgtttattctatatgaatattgtttcatgtttgtatgggaaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaaagcacaatgtgcagaagttatggaaaacctatagaggattacaacaggtaaacgttaaagagaatacattgctgacttatagtgatgtggctaagaagtacatgctttgttgtaaaattgcttgaaagcccattgaaagatgtatctgtttatttacagtctttgaagtaaaagttaccaatgtttgccaataaaaa

SEQ ID NO: 2 - protein, 326 aa, Homo sapiens
MHRTTRIKITELNPHLMCVLCGGYFIDATTIIECLHSFCKTCIVRYLETSKYCPICDVQVHKTRPLLNIRSDKTLQDIVYKLVPGLFKNEMKRRRDFYAAHPSADAANGSNEDRGEVADEDKRIITDDEIISLSIEFFDQNRLDRKVNKDKEKSKEEVNDKRYLRCPAAMTVMHLRKFLRSKMDIPNTFQIDVMYEEEPLKDYYTLMDIAYIYTWRRNGPLPLKYRVRPTCKRMKISHQRDGLTNAGELESDSGSDKANSPAGGIPSTSSCLPSPSTPVQSPHPQFPHISSTMNGTSNSPSGNHQSSFANRPRKSSVNGSSATSSG

SEQ ID NO: 3 - DNA, 21 bp, Artificial Sequence
  Sequence is completely synthesized
cctaatactttccagattgat

SEQ ID NO: 4 - DNA, 19 bp, Artificial Sequence
  Sequence is completely synthesized
ggtacttcattgatgccac

SEQ ID NO: 5 - DNA, 9 bp, Artificial Sequence
  Sequence is completely synthesized
ttcaagaga

SEQ ID NO: 6 - DNA, 19 bp, Artificial Sequence
  Sequence is completely synthesized
acgtgtattgttcgttacc

(19) United States
(12) Patent Application Publication
(10) Pub. No.: US 2019/0203254 A1
(43) Pub. Date: Jul. 4, 2019

(54) CORTICAL NEURON CELL MODEL OF ALZHEIMER'S DISEASE BASED ON BMI1 DEFICIENCY, AND USES THEREOF

(71) Applicant: 9636137 Canada Inc., Rosemère (CA)

(72) Inventors: Gilbert BERNIER, Rosemère (CA); Anthony Flamier, Montrèal (CA)

(21) Appl. No.: 16/322,545

(22) PCT Filed: Jun. 14, 2017
(86) PCT No.: PCT/IB2017/053544

§ 371 Date: Feb. 1, 2019

Related U.S. Application Data
(60) Provisional application No. 62/369,814, filed on Aug. 2, 2016.

(51) Int. Cl. C12Q 1/02; C12N 15/113; C12N 5/0793; C12N 5/074; C12N 5/0735; C12N 5/00

(52) U.S. Cl. CPC C12Q 1/025; C12N 15/113; C12N 5/0619; G01N 2800/2821; C12N 5/0606; C12N 5/0062; C12N 5/0696

(57) ABSTRACT

The invention is concerned with dementia-related neurological diseases and more particularly Alzheimer's disease. Herein described are primate cortical neuronal cells that are BMI1 -deficient and that displays one or more phenotypic hallmark of Alzheimer's disease. Also described are cellular models comprising such cells, methods for screening, designing anti-Alzheimer drugs and/or for identifying a potential biological target of an anti-Alzheimer drug using such cells. Described also are methods for diagnosing Alzheimer's disease, comprising assessing BMI1 activity and/or comprising detecting epigenetic BMI1 silencing.

### FIELD OF THE INVENTION

The invention relates to the field of dementia-related neurological diseases and more particularly to cells and methods for the development of therapies for Alzheimer's disease.

### BACKGROUND OF THE INVENTION

Sporadic AD (sAD) is the most common dementia with an estimated prevalence of 5.2 million Americans in 2014 {Hebert, 2013}. Total payments in 2014 for all individuals with sAD and other dementias are estimated at $214 billion in the US (American Alzheimer's Association 2014 report). Despite numerous clinical trials, there are actually no treatments to stop or delay sAD disease {De Strooper, 2014}. The brain changes in sAD begin 20 or more years before symptoms appear {Villemagne, 2013}. 11% of people aged 65 and older have sAD. 32% of people aged 85 and older have sAD. Thus, the greatest risk factor for sAD is advanced age {Sawa, 2009}. Carriers of the E4 allele of APOLIPOPROTEIN or of allelic variants of SORL1 have increased risk to develop AD {Kanekiyo, 2014}. In contrast, Familial AD (FAD) occurs between the ages of 30-50 year, is autosomal dominant and linked to mutation in APP, PSEN1 or PSEN2, representing less than 5% of AD cases {Blennow, 2006}. AD in general is characterized by progressive memory and behavioral impairment owing to degeneration of limbic and cortical areas of the brain. Pathological hallmarks of AD comprises the presence of amyloid plaques, neurofibrillary phospho-TAU tangles and synaptic dysfunction {Blennow, 2006}. Notably, the etiology of sAD has remained elusive and is still unknown.

Induced pluripotent stem (iPS) cells have been produced from skin fibroblasts of sAD patients. When differentiated into cortical neurons, some of the cell lines produced with this method display AD pathology in vitro {Israel, 2012}. Although these cells can also be used for drug screening assays, the screen cannot be directed toward novel AD-mediating pathway since the genetic or epigenetic origin of the disease remains unknown in these cell lines.

Accordingly, there is a need for a better understanding of the genetic etiology of dementia-related neurological diseases, including Alzheimer's disease and more particularly sAD, and a need for more accurate methods for diagnosing Alzheimer's disease and sAD.

There also is a need for valid experimental models of dementia-related neurological diseases, including Alzheimer's disease and sAD. Indeed a highly reproducible and robust in vitro human model of Alzheimer's disease and/or sAD could save millions of dollars to pharmaceutical industries.

There is more particularly a need for a highly reproducible and robust in vitro human sAD model in which the disease's genetic origin is known, in order to accurately test, screen and/or design novel therapeutics for sAD.

There is also a need for a highly reproducible and robust in vitro human cortical neuronal cell model of sAD in which the genetic origin of the disease is known, in order to validate (or invalidate) potential AD therapeutics which are being tested in phases I to III clinical trials.

The present invention addresses these needs and other needs as it will be apparent from review of the disclosure and description of the features of the invention hereinafter.

### BRIEF SUMMARY OF THE INVENTION

According to a first aspect, the invention relates to an isolated cell, wherein said cell is a primate cortical neuronal cell that is BMI1-deficient, and wherein said cell displays one or more phenotypic hallmark of Alzheimer's disease.

A related aspect of the invention concerns a cellular model of a dementia-related neurological disease, comprising an in vitro culture of a plurality of cortical neuronal cells as defined herein.

Another aspect of the invention concerns a method for screening anti-Alzheimer drugs, comprising:
exposing a cortical neuronal cell as defined herein to a candidate anti-Alzheimer compound;
assessing said cell for one or more phenotypic hallmark of Alzheimer's disease in presence of said candidate compound; and
selecting a candidate compound capable of inhibiting and/or reducing said one or one or more phenotypic hallmark of Alzheimer's disease.

Another aspect of the invention concerns a screening method for identifying a potential anti-Alzheimer drug, comprising (i) contacting a cell or animal having a BMI1-deficiency with a candidate compound to be tested; and (ii) assessing activity of said compound on one or more phenotypic hallmark of Alzheimer's disease.

According to another aspect, the invention relates to a the use(s) of a non-human animal having a BMI1-deficiency as an animal model of Alzheimer's disease.

The invention also relates to methods for designing anti-Alzheimer drugs and to methods for identifying a potential biological target of an anti-Alzheimer drug.

Another aspect of the invention concerns a method for diagnosing Alzheimer's disease in a subject, comprising assessing BMI1 activity in brain cells of said subject, wherein a reduced or absence of BMI1 activity is indicative of Alzheimer's disease.

A related aspect concerns a method of diagnosing sporadic Alzheimer's disease in a human subject, comprising detecting epigenetic BMI1 silencing in the hippocampus, in the frontal cortex and/or in the entorhinal cortex of said subject.

An advantage of the invention is that it provides strong evidence of the most likely genetic etiology of dementia in humans (e.g. Alzheimer's disease, frontotemporal dementia and dementia with Lewy Bodies), particularly the genetic etiology of Alzheimer's disease and more particularly sAD.

Another advantage of the invention is that it may be used for a rapid and powerful induction of sAD in human cortical neurons (in vitro) through a single genetic trigger, i.e. inactivation of BMI1.

Advantageously, the biological models of sAD according to the present invention may be used for small, medium and/or high throughput drug-screening and for validation (or invalidation) of drug candidates for dementia-related neurological diseases, including Alzheimer's disease and more particularly sAD. For instance, a reliable and relevant in vitro human model of sAD can potentially save millions of dollars to the pharmaceutical industry by allowing the validation of drug candidates against sAD before moving into very expensive clinical trials.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order that the invention may be readily understood, embodiments of the invention are illustrated by way of example in the accompanying figures.

FIG. 1 is a panel with pictures and graphs illustrating that Bmi1^{+/-} mice present a neuropathology resembling AD. FIG. 1A: Western blot of cortical extracts from 15-month old WT (n=3) and Bmi1^{+/-} mice (n=3). FIG. 1B: p-Tau immunoreactivity on cortical sections of 20-month old WT and Bmi1^{+/-} mice. (i) Low magnification. Scale bar: 20 μm. (ii) Higher magnifications. Arrowhead: p-TAU deposits on ghost-like neurons. (iii) p-Tau deposits in track fibers of the cortical white matter of Bmi1^{+/-} mice (arrowhead). Scale bar: 8 μm. FIG. 1C: Immunoprecipitation of amyloid (C99) of 15-month old WT and Bmi1^{+/-} cortices (soluble fraction) using DE2B4 antibody and revealed with the FCA3542 antibody. FIG. 1D: b-amyloid (DE2B4) immunoreactivity on cortical sections of 20-month old WT and Bmi1^{+/-} mice. Top: low magnification. Scale bar: 20 μm; Bottom: high magnification. Scale bar: 8 μm. FIG. 1E: Increased amyloid (C99), p-Tau (AT8) and p-JNK immunoreactivity in cortical extracts of 15-month old Bmi1^{+/-} but not in 24-month old WT mice. FIG. 1F: Decreased number of

NeuN+cortical neurons and increased number of activated caspase-3 cortical neurons in the frontal cortex of 15-month old WT (n=5) and Bmi1^{+/-} (n=5) using IHC. All values are mean ±SEM. (*) P<0.05; (**) P<0.01 by Student's unpaired t-test. FIG. 1G: Amyloid plaques (MOAB2) detection in the cortex (Ctx) and hippocampus (Hip) of 24-month old Bmi1^{+/-} mice but not in aged WT mice. Cb: cerebellum. FIG.

1H: Higher magnifications of amyloid-positive areas observed in FIG. 1G.

FIG. 2 is a panel with schemas, pictures and graphs illustrating that BMI1 deficiency in neurons induces a gene-expression signature related to AD. FIG. 2A: Protocol for the differentiation of human embryonic stem cells (hES) into cortical neurons and BMI1 knockdown. FIG. 2B: Confocal immunofluorescence of BMI1 and bill-tubulin of differentiated neurons infected with the shScramble (shCTL) or shBMI1 (shBMI1) viruses 7 days prior to analysis. Scale bar 40 mm. FIG. 2C: Reduced levels of BMI1 and H2Aub in shBMI1 neurons compared to shCTL neurons by immunoblot. FIG. 2D: Top: phase contrast images of shCTL and shBMI1 cortical neurons. White arrows indicate dystrophic neurites. Bottom: expression of the neuronal markers GABA and vGLUT1 (day 14). Scale bar 40 mm. FIG. 2E: Expression of the neuronal markers GABA and MAP2 in shCTL and shBMI1 neurons (day 14). White arrows: distal axonal atrophy. Scale bar 40 mm. FIG. 2F: Expression of the neuronal markers vGLUT1, bIII-tubulin and NeuN in shCTL and shBMI1 neurons (day 14). White arrows: axonal swelling and signal breaks along axonal segment. Scale bar 40 um. FIG. 2G: Quantification of GABA- and vGLUT1-positive neurons in shCTL (n=4) and shBMI1 (n=4) cultures. All values are mean±SEM. (*) P<0.05; by Student's unpaired t-test. FIG. 2H: Proportion of pyramidal neurons in the cultures (day 38). FIG. 2I: Normalized gene expression heatmap of hESC (n=3), shCTL neuorns (n=3) and shBMI1 neurons (n=3) for neuronal, Meso/endodermal and pluripotency markers. Top: color code legend for expression level. FIG. 2J: Left: volcano plot from ANOVA statistical analysis of shBMI1 versus shCTL neurons. Top right corner shows the significantly up-regulated genes in shBMI1 neurons. Right: gene ontology (PANTHER) analysis of up-regulated genes.

FIG. 3 is a panel with pictures and graphs illustrating that BMI1 deficiency in neurons results in p-Tau and amyloid accumulation. FIG. 3A: p-Tau (PHF1) accumulation in shBMI1 neurons but not in shCTL neurons (day 14). Scale bar: 40 mm. FIG. 3B: p-Tau (PHF1) accumulation in broken axonal segments of glutamatergic shBMl1 neurons (arrows), and co-localization of p-Tau with vGLUT1 in axonal swellings (arrows in the inset). Scale bar: 40 mm. FIG. 3C: Quantification of PHF1-positive neurons and of p-Tau foci/axon in shCTL (n=3) and shBMI1 (n=3) cultures. All values are mean±SEM. (*) P<0.05; (**) P<0.01 by Student's unpaired t-test. FIG. 3D: Confocal immunofluorescence for the apoptosis marker activated caspase-3 in shCTL and shBMl1 cultures. Scale bar: 40 mm. FIG. 3E: Quantification of activated caspase-3 positive cells in GABA+ and GABA− neuronal populations. All values are mean±SEM. (*) P<0.05 by Student's unpaired t-test. FIG. 3F: Amyloid accumulation in shBMl1 neurons as compared to shCTL neurons using the MOAB2 or PA3 antibodies. Scale bars: 40 mm. FIG. 3G: Confocal immunofluorescence for oligomeric amyloid (NU1) and Ab42 (PA3) of shCTL and shBMI1 neurons treated with beta-secretase inhibitor (BSI) or vehicle control. FIG. 3H: Intracellular and extracellular Ab42 levels in shCTL (n=3) and shBMl1 (n=3) neurons by ELISA. Right: Ab42 levels in shCTL (n=3) and shBMl1 (n=3) neurons treated with BSI or vehicle control. All values are mean±SEM. (**) P<0.01 by Student's unpaired t-test.

FIG. 4 is a panel with schemas, pictures and graphs illustrating that BMI1 deficiency in neurons results in p-Tau tangles and amyloid plaques formation. FIG. 4A: Experimental scheme for the long-term culture of cortical neurons in 3D. FIG. 4B: Reduced immunoreactivity for synaptic markers Synaptophysin (SYN) and PSD95 in shBMI1 neurons. Scale bar: 40 μm. FIG. 4C: Amyloid plaques formation in shBMI1 neurons using MOAB2 or the auto-fluorescent compound K114. Oligomeric amyloid deposition in shBMI1 neurons cytoplasm using NU1 antibody. Scale bar: 40 μm. FIG. 4D: Accumulation of p-Tau (PHF1 and 764G) as diffuse extra-cellular aggregates (i) or tangles (ii) in shBMI1 neurons in contrast to shCTL neurons. C₀-staining with cholinergic neurons marker ChAT reveals degenerating ChAT-positive shBMl1 neurons with large p-Tau tangles. Scale bar: 40 μm. FIG. 4E: Three-dimensional z-stacks reconstruction of p-Tau tangles located in shBMl1 neurons soma. FIG. 4F: Accumulation of aggregated amyloid (30 kDa), C99 fragment (14 kDa) and Aβ42 peptide (4kDa) in shBMI1 neurons lysates. FIG. 4G: Extracellular Ab42 levels in shCTL (n=4) and shBMI1 (n=4) neurons by ELISA. All values are mean±SEM. (***) P<0.001 by Student's unpaired t-test.

FIG. 5 is a panel with pictures and graphs illustrating that BMI1 deficiency in neurons results in activation of MAPT, GSK3b and p53. FIG. 5A: Immunoblots for AD-related proteins in shCTL, shBMI1 and BMI1 overexpressing (BMI1 OE) neurons. Protein expression ratios were established using Ponceau staining and where expression in shCTL neurons was set as 1. FIG. 5B: Confocal analysis of F-Actin (phalloidin), amyloid (PA3), p-Tau (PHF1) and activated caspase-3 (Casp3a) in shBMI1 neurons treated with DMSO, β-secretase inhibitor (BSI), γ-secretase inhibitor (GSI) or y-secretase modulator (GSM). Quantification was established using shCTL neurons set as 1. Scale bar: 40 μm.

| | | | | Working | |
|---|---|---|---|---|---|
| Compound | Company | Name | Catalog # | concentration | |
| BSI | SCB | BSI-IV | sc-222304 | 1 | μM |
| GSI | Sigma | DAPT | D5942 | 0.5 | μM |
| GSM | Millipore | GSM XXII | 565791 | 30 | μM |

FIG. 6 is a panel with schemas, pictures and graphs illustrating that BMI1 brain expression is reduced in sAD but not in other related dementias. FIG. 6A: BMI1 gene expression levels in qPCR normalized to GAPDH, FOXG1, ASCL1 and NEUROD1 in the hippocampus of CTL (n=2) and AD patients (n=3). Top: percentage of BMI1 reduction in AD versus CTL brains. All values are mean±SEM. (*) P<0.05; (**) P<0.01; by Student's unpaired t-test. FIG. 6B: BMI1 gene expression levels in qPCR normalized to GAPDH using 4 non-overlapping primer pairs in the hippocampus of CTL (n=2) and AD patients (n=3). All values are mean±SEM. (*) P<0.05; (**) P<0.01; by Student's unpaired t-test. FIG. 6C: BMI1 gene expression levels in qPCR normalized to GAPDH in the hippocampus of CTL (n=5) and AD patients (n=6) from two independent sources (Douglas hospital and Banner health institute). (**) P<0.01; by Student's unpaired t-test. FIG. 6D: EZH2 gene expression levels in qPCR normalized to GAPDH in the hippocampus of CTL (n=5) and AD patients (n=6) from two independent sources (Douglas hospital and Banner health institute). FIG. 6E: Immunostaining for BMI1 (black arrows) and NeuN (small black squares) on frontal cortex sections from age-matched control and AD patient. Note lipofuscin deposition (small stars). Scale bar: 8 μm. FIG. 6F: Immunoblot on hippocampal extracts from young control (n=3), FAD (n=4) and AD (n=2) patients using clone F6 against BMI1. FIG. 6G: Immunoblot on frontal cortex from control (n=6) and AD (n=6) patients using clone F6 against BMI1. FIG. 6H: Quantification of BMI1 protein levels in control, AD, fronto-temporal dementia (FTD), dementia with Lewy body (DLB), Pick's disease (PSP), Korsakoffs syndrome (KS) and DLB with synuclein patients. All values are mean±SEM. (**) P<0.01; by one-way ANOVA. FIG. 61: ChIP-qPCR on control (#428; 89y) and AD (#1127: 88y) frontal cortex samples using IgG, H2A^{ub} and Bmi1 clone 1.T.21 (ab14389) antibodies. FIG. 6J: Immunoblot on hippocampal extracts from control (n=2) and AD (n=2) patients. FIG. 6K: Quantification of BMI1 and H2Aub protein levels in hippocampal (Hippo) and frontal cortex (Fct) extracts from control, FAD, and AD patients. Hippocampus: 3 young controls, 4 FAD, 3 old controls and 5 AD brains. Frontal cortex: 6 old controls and 6 AD brains. All values are mean±SEM. (*) P<0.05; (**) P<0.01; (***) P<0.001 by Student's unpaired t-test.

FIG. 7 is a panel with pictures illustrating that BMI1 brain expression is reduced in sAD but not in other related dementias using an independent antibody. FIG. 7A: Immunoblot on WT and Bmi1 cortical extracts using Cell signaling BMI1 antibody clone D42B3. FIG. 7B: Immunoblot on frontal cortex from control (n=4) and AD (n=5) patients using clone D42B3 BMI1 antibody. FIG. 7C: Immunoblot on frontal cortex from control, AD, fronto-temporal dementia (FTD), Pick's disease (PSP) and Korsakoffs syndrome (KS) patients using clone D42B3 BMI1 antibody. FIG. 7D: Immunoblot on frontal cortex from control, AD and dementia with Lewy body (DLB) patients using clone D42B3 BMI1 antibody.

FIG. 8 is a panel with pictures illustrating that BMI1 loss induces chromatin anomalies also found in AD brains. FIG. 8A: Western blot of cortical extracts from 15-month old WT (n=3) and Bmi1^{+/-} mice (n=3). FIG. 8B: Immunofluorescence of cortical extracts from 15-month old WT and Bmi1^{+/-} mice for H3K9me3. FIG. 8C: Immunofluorescence of shCTL and shBMI1 human cortical neurons for H3K9me3. FIG. 8D: Immunoblot on frontal cortex from control (n=6) and AD (n=6) patients using H3K9me3 antibody. FIG. 8E: Immunostaining for H3K9me3 on frontal cortex sections from age-matched control and AD patient. FIG. 8F-G: Quantification of picture in E. All values are mean±SEM. (*) P<0.05; (**) P<0.01 by Student's unpaired t-test.

FIG. 9 is a series of panels illustrating that BMI1 knockout in human neurons results in AD pathologies. FIG. 9A: Schematic representation of the experimental procedure for BMI1 inactivation in post-mitotic neurons. For panels B-F, cortical neurons were transfected three times with a plasmid for the expression of the CRISPR/Cas9 nuclease, a synthetic scramble guide RNA (sgRNA-Ctrl) or a guide RNA complementary to BMI1 Exon1 (sgRNA-BMI1) after 1 month of maturation. Transfected neurons were analyzed after 2 weeks in culture. For panels G-H, cortical neurons were transfected three times after 2 months of maturation and analyzed after a week in culture. FIG. 9B: Panel showing a picture of genomic PCR on wild-type (sgRNA-Ctrl) and BMI1 null (sgRNA-BMI1) neurons at 4 different localizations of

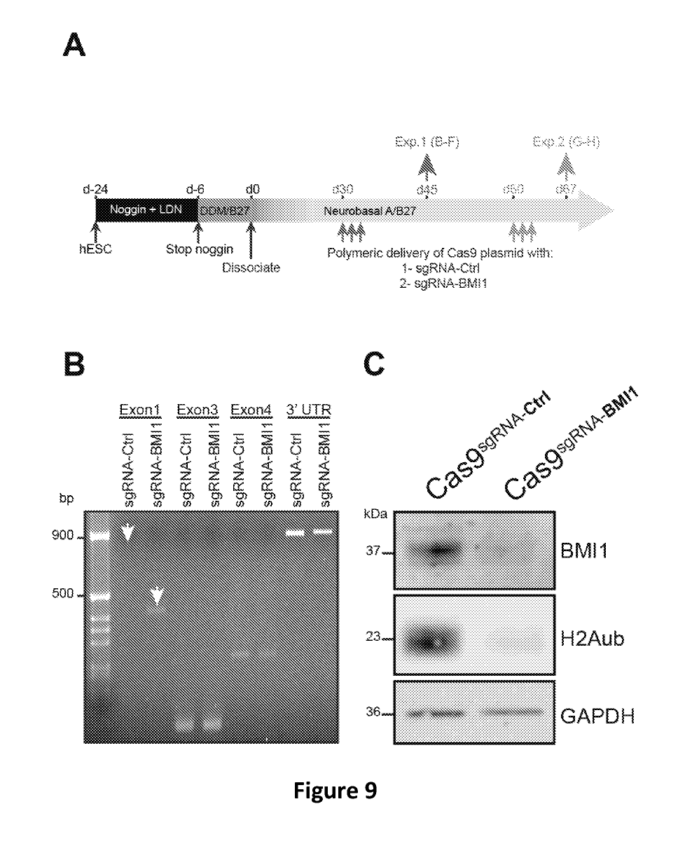

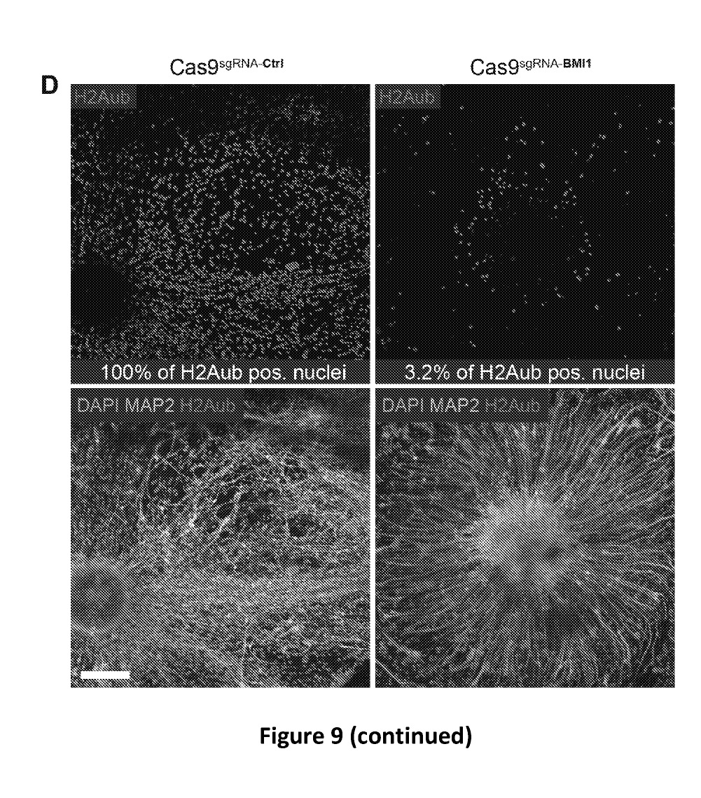

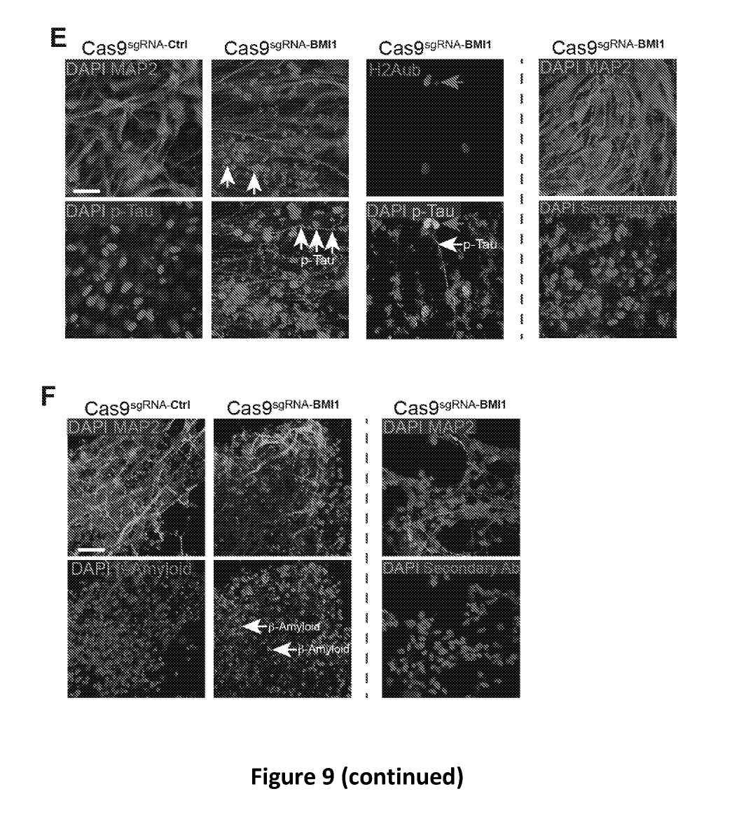

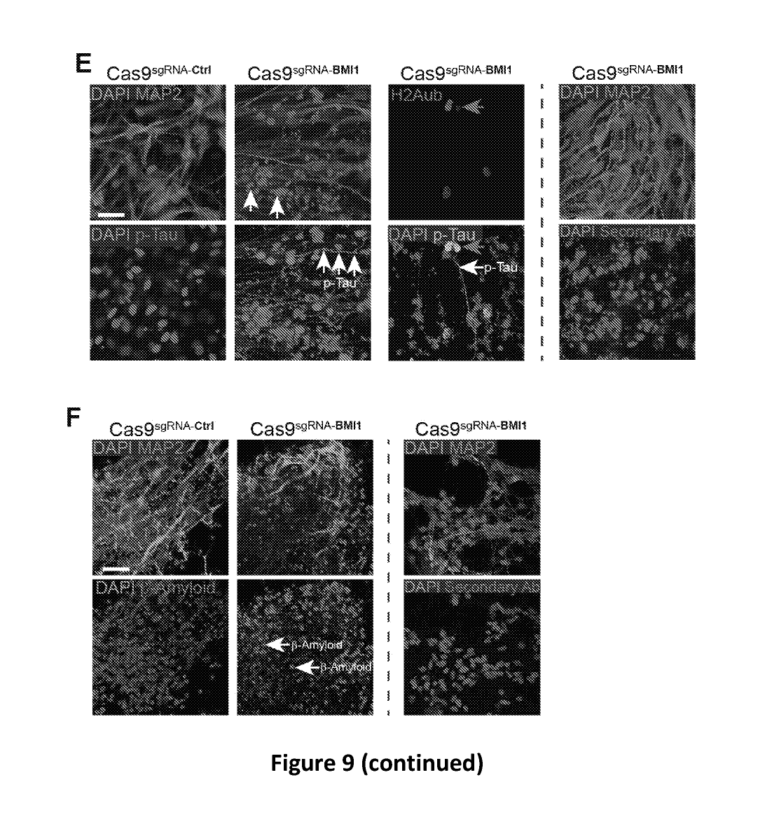

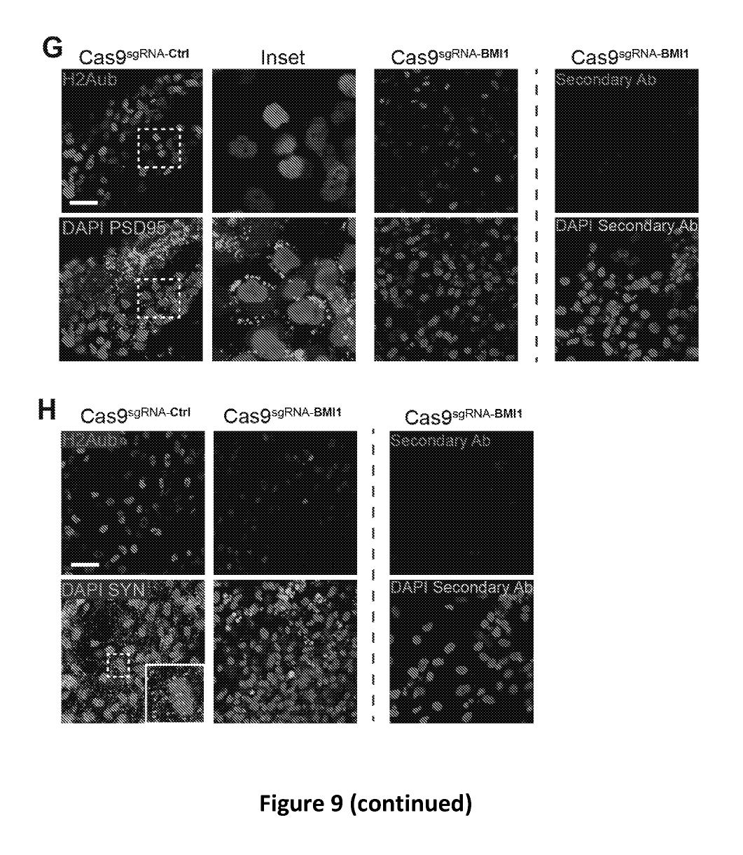

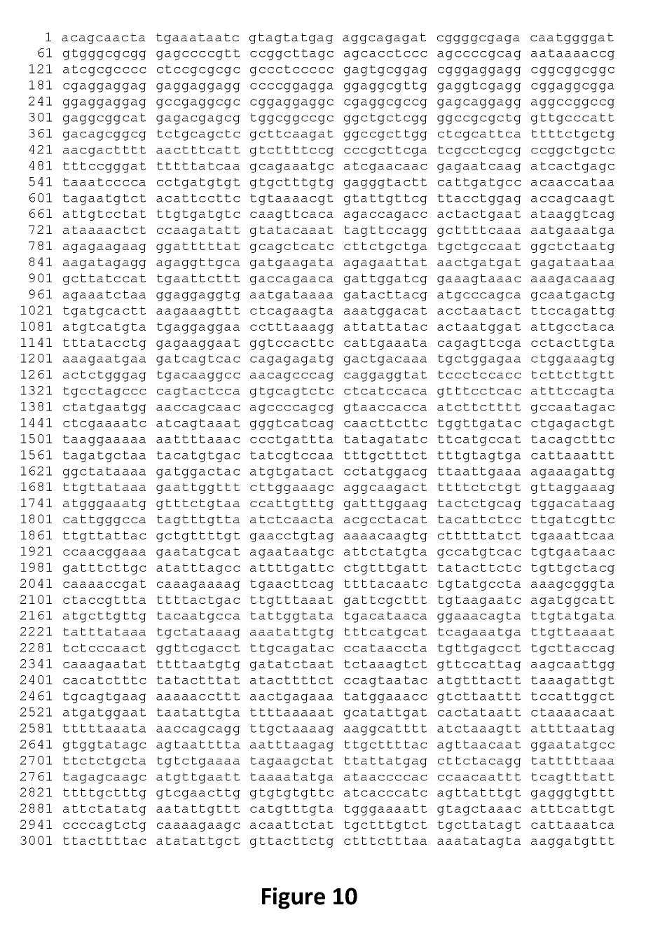

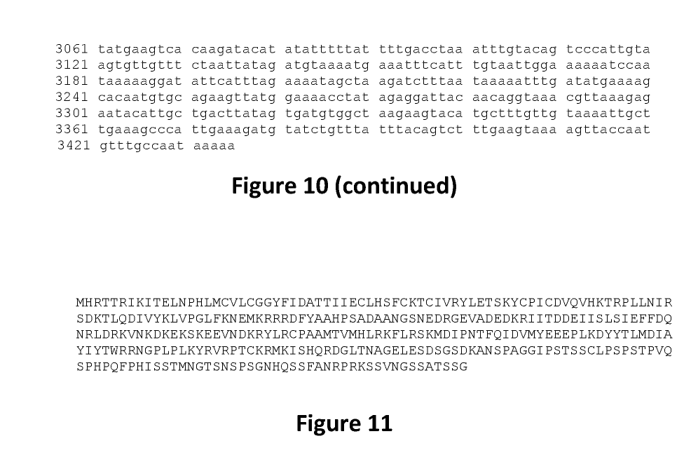

BMI1 locus. Note the deletion on Exon1 (white arrows) but not in Exon3, Exon4 or 3′ end of the gene. FIG. 9C: Panel showing pictures of immunoblot on sgRNA-Ctrl and sgRNA-BMI1 neurons for BMI1 and H2Aub. FIG. 9D: Panel showing pictures of immunofluorescence on sgRNA-Ctrl and sgRNA-BMI1 neurons for MAP2 and H2Aub. Note the reduction of H2Aub positive nuclei in sgRNA-BMI1 neurons. Scale bar: 100 □m. FIG. 9E: Panel showing pictures of p-Tau (PHF1) accumulation in sgRNA-BMI1 but not sgRNA-Ctrl neurons. Note the presence of piknotic nuclei in sgRNA-BMI1 (white arrows) and the accumulation of p-Tau specifically in neurons KO for BMI1 (red arrow). sgRNA-BMI1 neurons stained using the secondary antibody only was used to assay for possible non-specific background fluorescence. FIG. 9F: Panel showing pictures of amyloid (D9A3A) accumulation in sgRNA-BMI1 but not sgRNA-Ctrl neurons. sgRNA-BMI1 neurons stained using the secondary antibody only was used to assay for possible non-specific background fluorescence. FIG. 9G: Panel showing pictures of reduced immunoreactivity for post-synaptic marker PSD95 in sgRNA-BMI1 but not sgRNA-Ctrl neurons. sgRNA-BMI1 neurons stained using the secondary antibody only was used to assay for possible non-specific background fluorescence. FIG. 9H: Panel showing pictures of reduced immunoreactivity for pre-synaptic marker Synaptophysin (SYN) in sgRNA-BMI1 but not sgRNA-Ctrl neurons. sgRNA-BMI1 neurons stained using the secondary antibody only was used to assay for possible non-specific background fluorescence. (E-H) Scale bar: 40 μm.

FIG. 10 provides the genetic sequence of BMI1 (SEQ ID NO: 1). The coding sequence extends from 507-1487.

FIG. 11 provides the amino acid sequence of BMI1 (SEQ ID NO: 2).

Further details of the invention and its advantages will be apparent from the detailed description included below.

### DETAILED DESCRIPTION OF PREFERRED EMBODIMENTS

In the following description of the embodiments, references to the accompanying drawings are by way of illustration of an example by which the invention may be practiced. It will be understood that other embodiments may be made without departing from the scope of the invention disclosed.

### General Overview

To date, the genetic etiology of sAD is unknown and, as such, there is no valid experimental models of sAD. The present inventors provide strong evidence that a single genetic trigger, i.e. inactivation of BMI1, is the most likely genetic etiology of dementia in humans (e.g. Alzheimer's disease, frontotemporal dementia and dementia with Lewy Bodies), particularly the genetic etiology of Alzheimer's disease and more particularly sAD.

The inventors have obtained an inducible genetic model of late-onset sporadic Alzheimer's disease (sAD) in human cortical neurons. According to one embodiment, it is possible to generate large amounts of human cortical neurons using human pluripotent stem cells or human embryonic stem cells. The post-mitotic neurons or their progenitors are next infected with a lentivirus expressing a small hairpin RNA against BMI1 (shBMI1). Similar results can be obtained using multiple plasmid DNA transfections of the shBMI1 construct. After only 2 weeks of BMI1 knockdown, it is possible to see a neuronal phenotype displaying hallmarks of sAD: axonal swelling, axonal segment breaks, beta-amyloid and p-Tau accumulation, and neuronal apoptosis.

The present application describes methods on how to inactivate BMI1 in human cortical progenitors/neurons produced from the differentiation of human embryonic stem cells. Results reveals that upon loss of BMI1, human cortical neurons display several anomalies such as axonal swelling, axonal segment breaks, and a gene-expression signature highly related Cadherin/Wnt and Alzheimer's disease/Presenilin signaling.

The present inventors have also observed the formation of F-Actin bundles in shBMI1-treated neurons using Phalloidin staining. This phenotype is thought to be secondary to p-Tau or total Tau accumulations. Likewise, upon long-term (6-8 weeks) neuronal cultures in Matrigel™ (i.e. 3D culture), the inventors have observed formation of extra-cellular amyloid plaques, intra-cellular p-Tau tangles, and pre- and post-synaptic atrophy and/or degeneration. The cell model according to the present invention thus recapitulates hallmarks of sAD. This also extends to the gene expression profile of shBMI1 neurons, which present the signature of sAD.

In particular embodiments, the present invention features methods wherein sAD modeling can be induced in human cortical neurons (likely from any sources) through genetic inactivation (e.g. CRISPR/Cas9 and all equivalent gene-editing methods or CRE/Ioxp mediated genetic recombination) or gene knockdown (shRNA or siRNA) of a single factor i.e. BMI1.

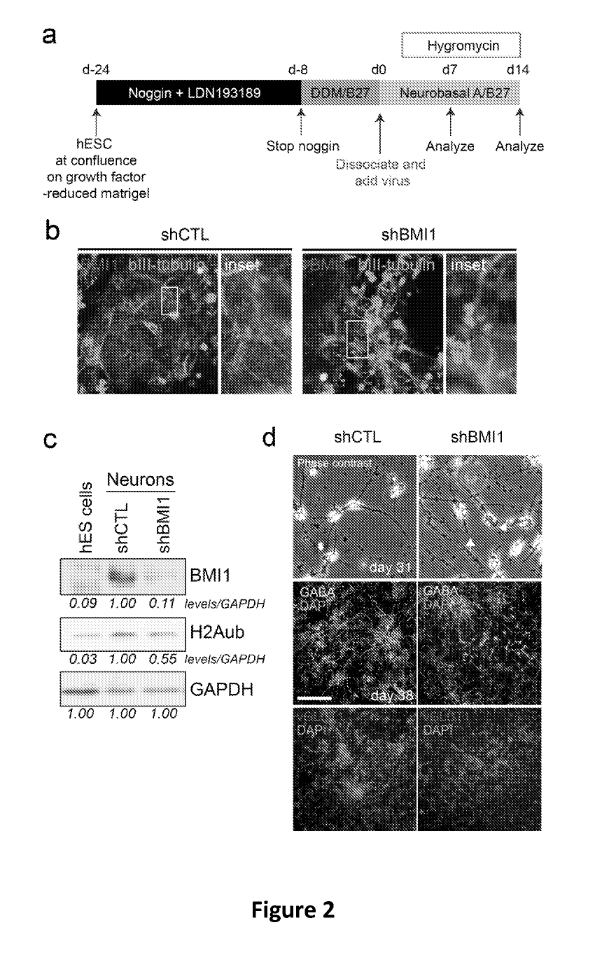

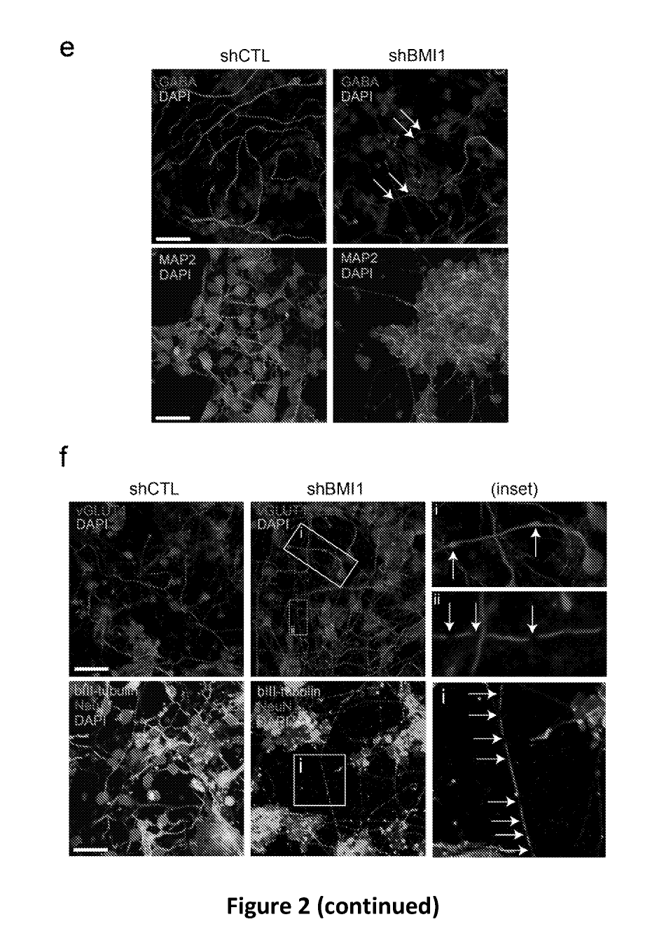

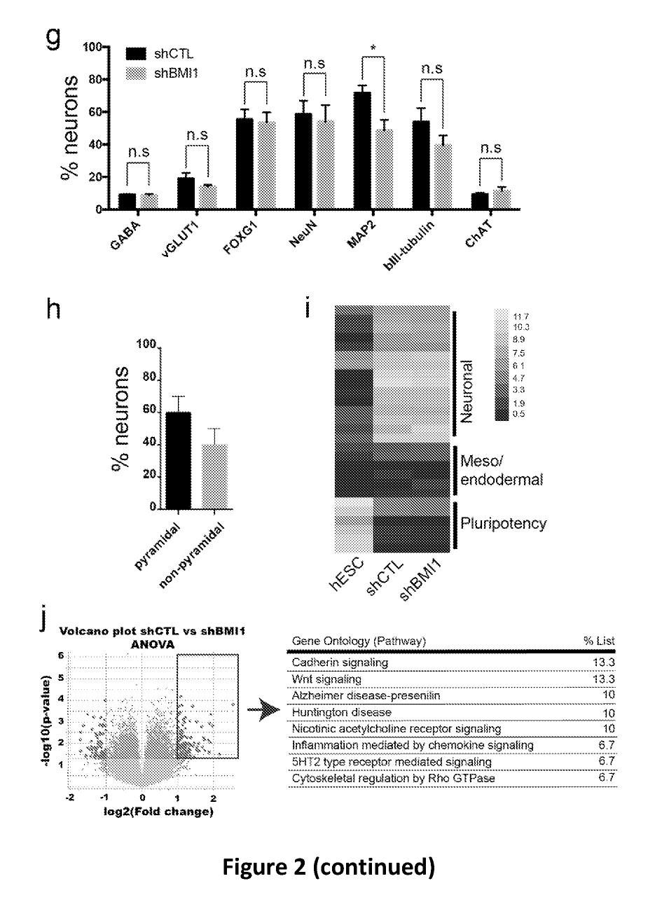

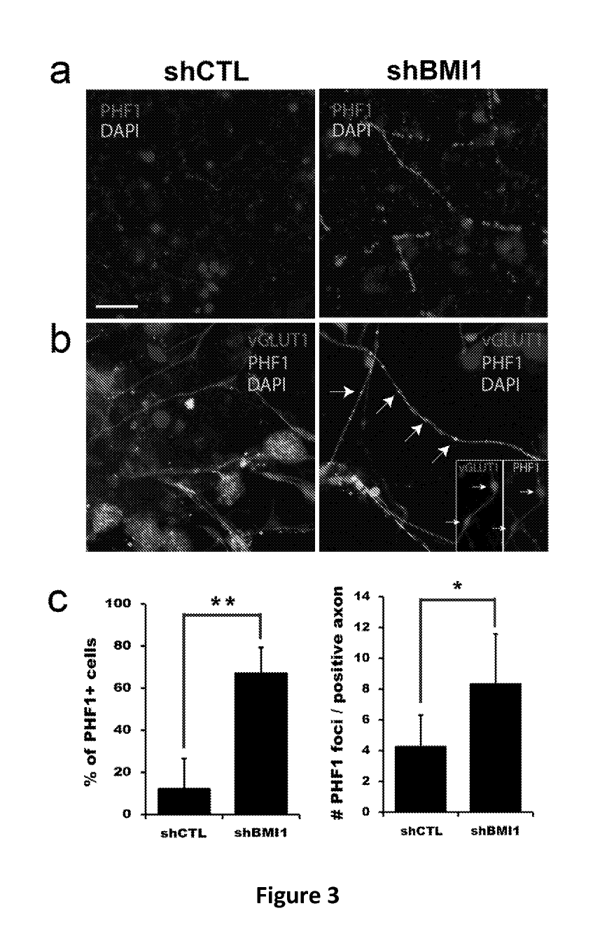

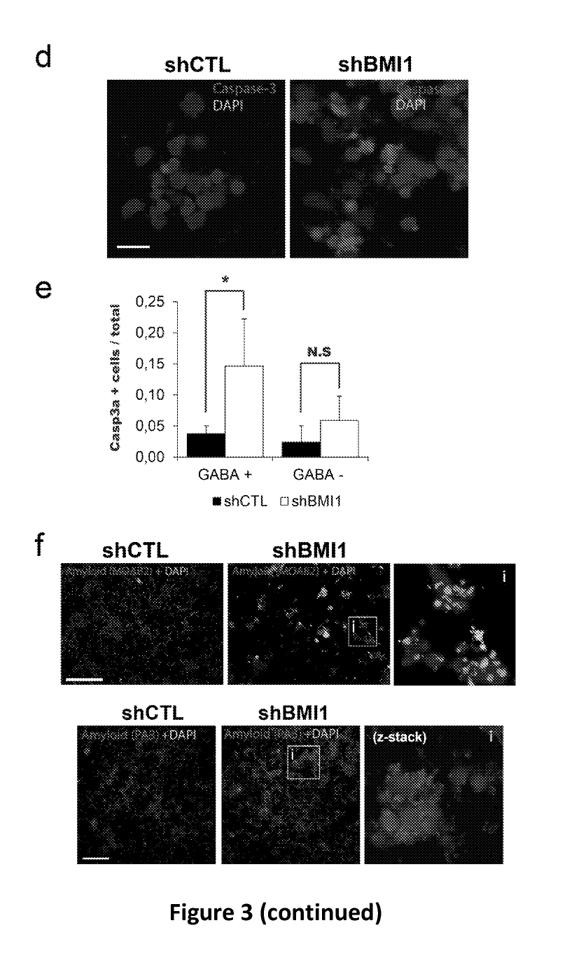

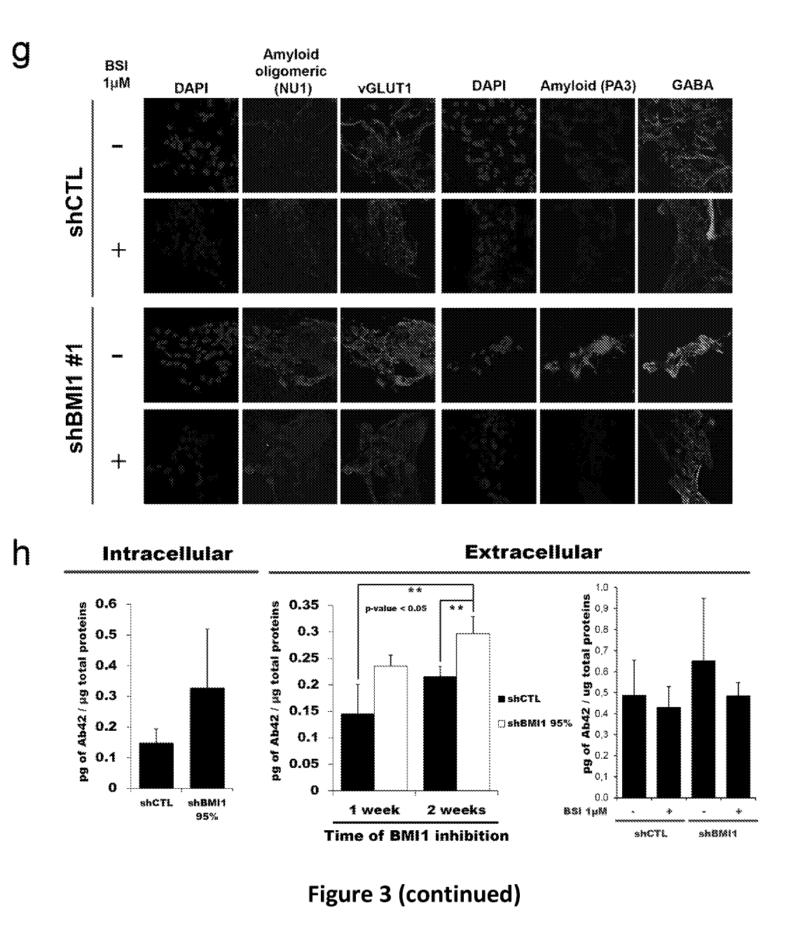

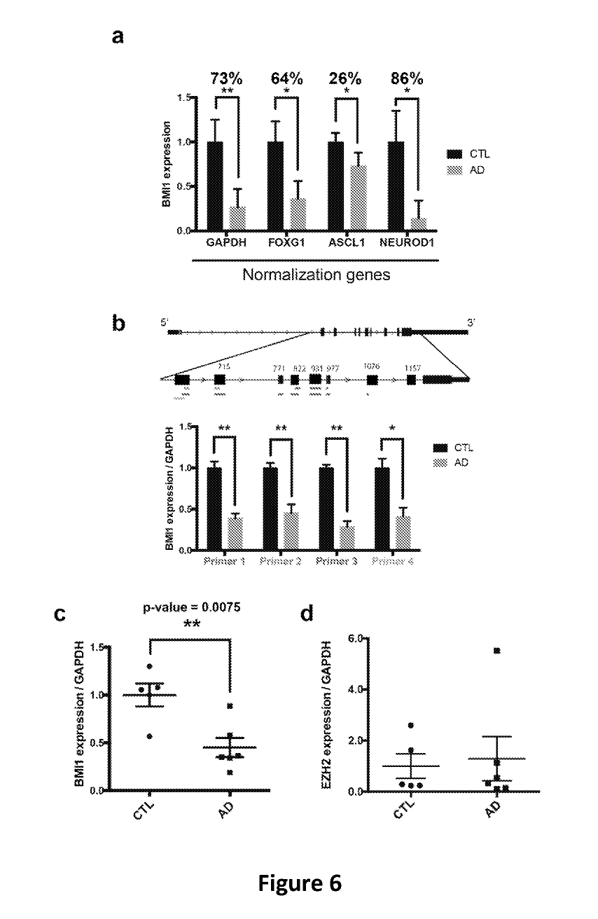

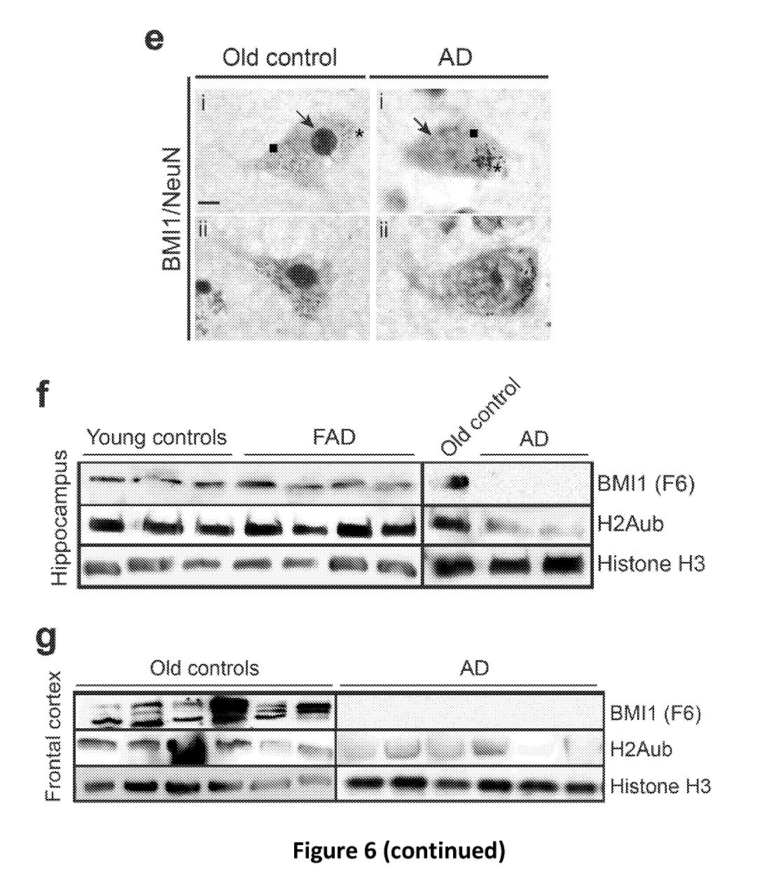

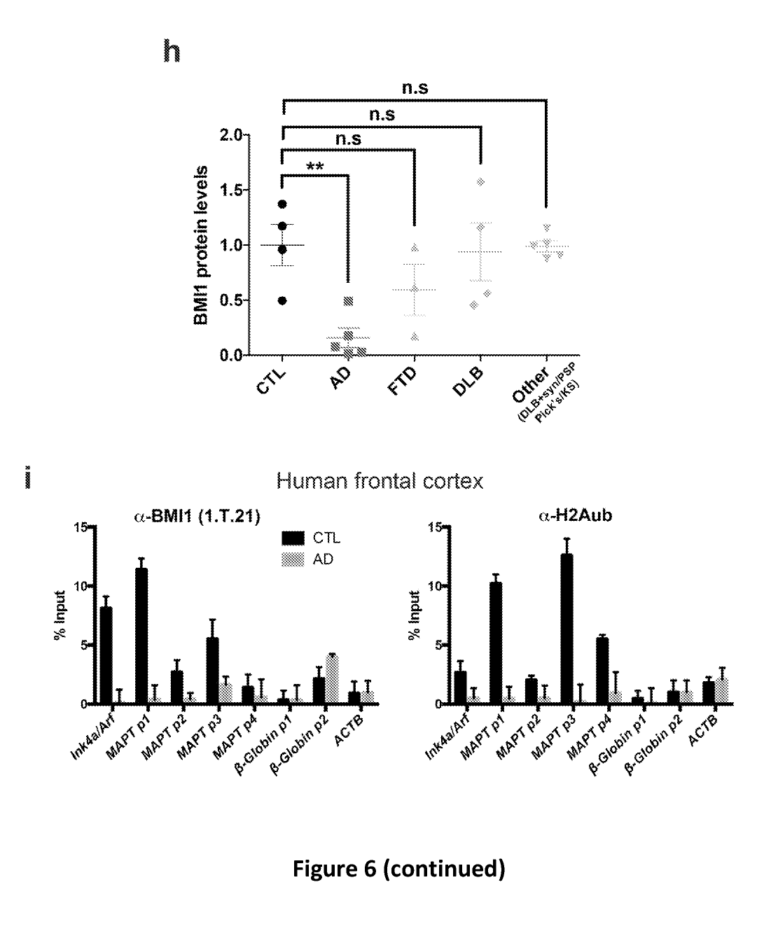

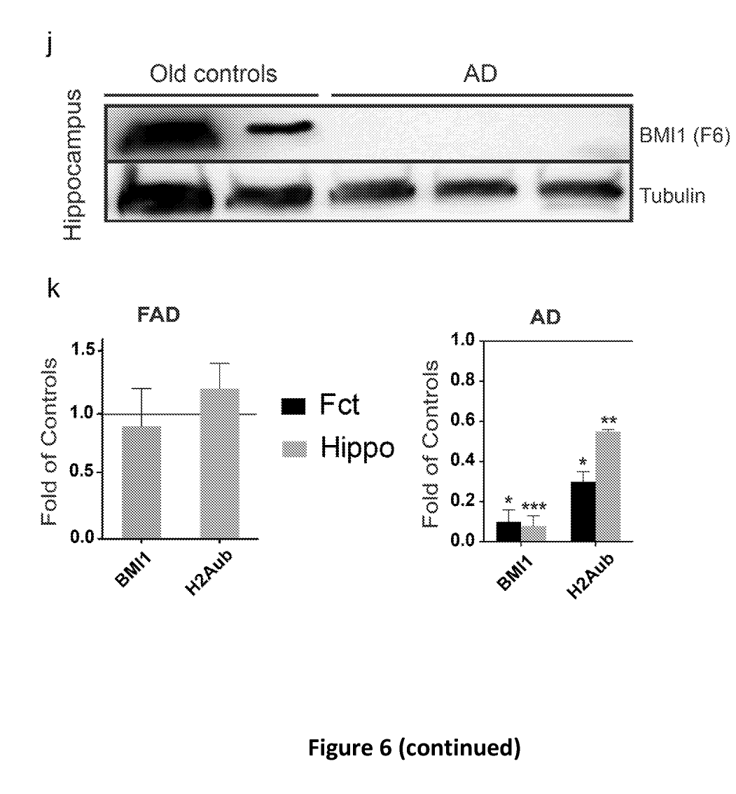

In addition, in embodiments the present invention features methods wherein it is possible to induce a very rapid and robust (thus easily measurable) disease phenotype in 15 days (FIGS. 2-3), thereby allowing directed drug targeting of molecular pathways regulated by BMI1 (FIGS. 2i-j and 5i). Also, because BMI1 expression is deficient in nearly all sAD brain samples analyzed to date (FIG. 6), this suggests that drugs capable of rescuing the in vitro sAD phenotype will be efficient in most sAD patients.

According to the present invention, a genetic “loss-of-function” of BMI1 gene recapitulates sAD hallmarks in human cortical neurons, including cholinergic neurons. Accordingly, the invention represents a unique tool for drug screening and drug validation assays. It also represents a unique cell model for understanding the molecular pathway(s) and cascade(s) leading to sAD for rationale design of new dementia-related therapies.

### Isolated BMI-Deficient Cells

According to one aspect, the invention relates to isolated cells that are BMI1-deficient and which display one or more phenotypic hallmark of Alzheimer's disease.

As used herein, the term “BMI1” generally refers to the Polycomb complex protein BMI-1 also known as polycomb group RING finger protein 4 (PCGF4) or RING finger protein 51 (RNF51), a protein that in humans is encoded by the BMI1 gene (B cell-specific Moloney murine leukemia virus integration site 1).

As is known, BMI1 is a protein of about 37-42 kDa. The GenBank™ accession number of human cDNA sequence of BMI1 is NM_005180.8 and is cited under NCBI Gene ID:648. FIG. 9 provides the genetic sequence of BMI1 (SEQ ID NO: 1). The coding sequence extends from 507-1487. FIG. 10 provides the amino acid sequence of BMI1 (SEQ ID NO: 2). In humans, there is a yet uncharacterized isoform generated by an upstream gene promoter element, that isoform generating a ˜70 kDa protein (COMMD3-BMI1).

As used herein, the term “BMI1-deficient”, or a related term such as “BMI1-deficiency”, refers to repression, inactivation, inhibition, etc. of BMI. In may refer for instance to repression, inactivation and/or inhibition of BMI1 gene expression, BMI1 protein expression and/or BMI biological activity. In embodiments it refers to situations where BMI1 has been inactivated, deleted or its expression has been knockdown at the genetic level. In some embodiments, it encompasses situations where BMI1 biological activity has been suppressed with a pharmaceutical inhibitor. In some embodiment, the term may encompasses repression, inactivation, inhibition, etc. of active isoform(s) of BMI.

In preferred embodiments, the cell is a primate cortical neuronal cell. As used herein, the term “primate” encompasses human and non-human primates, including but not limited to chimpanzee, monkey, macaques and gorilla. Preferably, the primate is a human.

As used herein, the term “Alzheimer's disease” or “AD” encompasses both, the sporadic and the genetic (i.e. familial) forms of Alzheimer's disease. In preferred embodiments, the invention is directed to the sporadic Alzheimer's disease (sAD), i.e. cells that represent a valid and robust model of sAD.

In embodiments, the cell of the invention displays one or more phenotypic hallmark of Alzheimer's disease selected from: axonal swelling, axonal segment breaks, beta-amyloid accumulation, C99 fragment accumulation, p-Tau accumulation, synaptic atrophy, neuronal apoptosis, heterochromatin relaxation using H3K9me3 antibody, F-Actin bundles formation using Phalloidin staining, and combinations thereof.

A BMI1-deficient primate cortical neuronal cell according to the present invention may be obtained using any suitable method or technology. For instance, it may be possible to inactivate BMI in a variety of cells. For instance, a BMI1-deficient primate cortical neuronal cell may be obtained and/or it may derive from various source such as primary neuronal tissue from human foetus, post-mortem human brain tissue, human neural stem cells, or induced neurons from human somatic cells. In preferred embodiments, the BMI1-deficient primate cortical neuronal cell according to the present invention is a human induced pluripotent stem (iPS) cell or a human embryonic stem cell.

Also, repression, inactivation and/or inhibition of BMI1 gene expression, BMI1 protein expression and/or BMI biological activity may be achieved using any suitable method or technology. For instance, BMI1 may be inactivated through genetic inactivation using one or more of: a micro-RNA against the BMI1 gene, a siRNA against the BMI1 gene, a shRNA against the BMI1 gene, a genetic inactivation of the BMI1 gene using CRE-Ioxp, a genetic inactivation of the BMI1 gene using CRISPR/Cas9 (e.g. see Example 9), a genetic inactivation of the BMI1 gene using TALEN, a genetic inactivation of the BMI1 gene using ZFNs, or any other suitable gene-editing or homologous recombination methods, and combinations thereof. In preferred embodiments, the BMI-deficient cortical neuronal cells are obtained by knockdown or by genetic deletion of BMI1 in human cortical neurons. In embodiments, BMI biological activity is repressed, inactivated and/or inhibited using a pharmaceutical inhibitor of BMI1, for instance PTC Therapeutics' BMI1 inhibitors PTC-209 (available from Sigma cat#SML1143) and PTC-596 (ongoing clinical trials for cancer therapy, see PTC Therapeutics' web page).

Accordingly, related aspects of the invention concern methods of preparing the cell of the invention. In one embodiment the method comprising the steps of: (i) providing primate cortical neuronal cells; and (ii) inactivating or suppressing BMI1 expression or biological activity.

### Cellular Model of Dementia-Related Neurological Diseases

Another aspect on the present invention concerns a cellular model of a dementia-related neurological disease, the model comprising an in vitro culture of a plurality of BMI-deficient cortical neuronal cells as defined herein.

In one embodiment, the BMI-deficient cortical neuronal cells are cultured in three dimensions using any suitable matrix including, but not limited to a 3D Matrigel™ matrix.

The cellular model may also comprises other cells that are co-cultured with the BMI-deficient cortical neuronal cells. Examples of such other cells include for instance astrocytes. In one embodiment, the BMI-deficient cortical neuronal cells are co-cultured with astrocytes as supporting cells. In embodiments the cortical neuronal cells are human cells and the astrocytes are mammalian cells (e.g. human, primate, mouse, rat, etc.). In preferred embodiments, the cortical neuronal cells are the astrocytes are both human cells.

The cellular model may be useful in connection with any dementia-related neurological disease including, but not limited to, Alzheimer's disease, frontotemporal dementia and dementia with Lewy Bodies. In preferred embodiment, the dementia-related neurological disease is Alzheimer's disease (e.g. genetic or sporadic), more preferably sAD.

### Screening Methods

The cells and cellular model according to the present invention may be particularly useful for drug screening and drug validation assays. They may also represent a unique model for understanding the molecular pathway(s) and cascade(s) leading to dementia, including sAD, and for rationale design of new dementia-related therapies.

Accordingly, related aspects of the invention concern methods for screening drugs and molecule for the treatment of dementia-related disease (e.g. Alzheimer's disease, frontotemporal dementia and dementia with Lewy Bodies).

As used herein, reference to terms such as “a molecule for the treatment” or “a drug against” a particular disease (and/or any similar expression), encompass any molecule or compound (natural or chemically synthesized) that may be administered to a subject in need with the purpose of stabilizing, curing, healing, alleviating, relieving, altering, remedying, less worsening, ameliorating, improving, or affecting the disease or condition, the symptom of the disease or condition, or the risk of (or susceptibility to) the disease or condition.

According to one particular aspect, the invention concerns a method for screening anti-Alzheimer drugs. In one embodiment, the method comprises:
exposing a BMI1-deficient primate cortical neuronal cell as defined herein to a candidate anti-Alzheimer compound;
assessing said cell for one or more phenotypic hallmark of Alzheimer's disease in presence of said candidate compound;
selecting a candidate compound capable of inhibiting and/or reducing said one or one or more phenotypic hallmark of Alzheimer's disease.

According to another particular aspect, the invention concerns a screening method for identifying a potential anti-Alzheimer. In one embodiment, the method comprises: (i) contacting a cell or animal having a BMI1-deficiency with a candidate compound to be tested; and (ii) assessing activity of said compound on one or more phenotypic hallmark of Alzheimer's disease. Preferably, the cell is a BMI1-deficient primate cortical neuronal cell as defined herein. The animal may be any animal having a BMI1-deficiency or susceptible of having a dementia-related disease corresponding to human Alzheimer's disease (see hereinafter).

The phenotypic hallmark may be selected from axonal swelling, axonal segment breaks, beta-amyloid accumulation, C99 fragment accumulation, p-Tau accumulation, synaptic atrophy, neuronal apoptosis, heterochromatin relaxation using H3K9me3 antibody, F-Actin bundles formation using Phalloidin staining, and combinations thereof.

In embodiments, the assessing is carried out in presence or subsequent to said contacting or in presence or subsequent to said exposing with the candidate compound.

In embodiments, the selecting step comprises selecting a candidate compound having one or more of the following biological effects:
prevention or inhibition of axonal swelling;
prevention or inhibition of axonal segment breaks;
prevention or inhibition of beta-amyloid accumulation;
prevention or inhibition C99 fragment accumulation;
prevention or inhibition of p-Tau accumulation;
prevention or inhibition of synaptic atrophy;
prevention or inhibition of neuronal apoptosis;
prevention or inhibition of heterochromatin relaxation using H3K9me3 antibody;
prevention or inhibition of F-Actin bundles formation using Phalloidin staining; and
prevention or inhibition of nuclear membrane anomalies, for instance by using Lamin A/C antibody or LaminA/C-GFP, RFP, YFP, mTomato, mCherry or any fluorescent LaminA/C fusion protein.

In embodiments, the assessing step comprises comparing said one or more phenotypic hallmark with wild-type cortical neurons exposed or not to the candidate compound. In embodiments, the assessing step comprises comparing said one or more phenotypic hallmark with control cells or animals not exposed to the candidate compound.

Another aspect on the present invention concerns a kit for screening for screening drugs and molecules for the treatment of a dementia-related disease (e.g. anti-Alzheimer drugs). According to one embodiment the kit comprises a BMI1-deficient primate cortical neuronal cell as defined herein, and/or the cellular model as defined herein, and one or more additional components. Examples of possible additional components include, but are not limited to, assay buffer(s), controls, substrate(s), standards, detection materials (e.g. antibodies, fluorescein-labelled derivatives, luminogenic substrates, detection solutions, scintillation counting fluid, etc.), laboratory supplies (e.g. desalting column, reaction tubes or microplates (e.g. 96- or 384-well plates)), a user manual or instructions, etc.

Accordingly a related aspect of the invention relates to the uses of non-human animals having a BMI1-deficiency as an animal model of dementia-related diseases (e.g. Alzheimer's disease, frontotemporal dementia and dementia with Lewy Bodies). Although such animals may already exists, they have never been used for such purposes.

According to the present uses, the animal may be any non-human animal having a

BMI1-deficiency or that is susceptible of having a dementia-related disease corresponding to human Alzheimer's disease. For instance, the animal may display one or more phenotypic hallmark of Alzheimer's disease including, but not limited to, axonal swelling, axonal segment breaks, beta-amyloid accumulation, C99 fragment accumulation, p-Tau accumulation, synaptic atrophy, neuronal apoptosis F-Actin bundles formation using Phalloidin staining, and combinations thereof.

In embodiments, the non-human is a non-human mammal, preferably a non-human primate, which is hemizygous for BMI1. In embodiments, the animal is a rodent, such as the BMI-deficient mouse that is hemizygous for BMI1 (van der Lugt et al. 1994, Chatoo et al. 2009).

A non-human animal model of Alzheimer's disease according to the present invention may be useful at various levels. For instance, it may be used for studying Alzheimer's disease and/or for identifying, testing, screening and/or designing (e.g. perform rationale drug-design) potential anti-Alzheimer compounds.

As such, the cells, cellular models, kits and non-human animals according to the present invention may find various useful applications for instance: (1) for small, medium and/or high throughput drug-screening assays to identify new drugs against dementia-related neurological diseases, including Alzheimer's disease and more particularly sAD; (2) small, medium and/or high throughput drug-screening assays to validate or invalidate candidate drugs against dementia-related neurological diseases, including Alzheimer's disease and more particularly sAD; (3) for logical drug-design against dementia-related neurological diseases, including Alzheimer's disease and more particularly sAD, through molecular dissection of pathways regulated by BMI1, and/or of pathways dis-regulated after induction of BMI1 deficiency, by using genomic, epigenomic and/or proteomic technologies; (4) in toxicity and pharmacokinetic assays of candidate drugs against dementia-related neurological diseases, including Alzheimer's disease and more particularly sAD, on normal and BMI1-deficient human cortical neurons; (5) as a cellular platform to test gene-therapy systems against dementia-related neurological diseases, including Alzheimer's disease and more particularly sAD, such as Adeno-Associated Virus (AAV) expressing BMI1 or other candidate genes, antisense oligonucleotides (against MAPT, APP, p53 or GSK3b for example), micro-RNA, etc.

Accordingly, an addition aspect of the invention concerns a method for designing drugs for use against dementia-related neurological diseases, including Alzheimer's disease and more particularly sAD. According to one particular aspect, the invention concerns a method for designing anti-Alzheimer drugs, the method comprising the steps of:
(a) exposing a BMI1-deficient primate cortical neuronal cell as defined herein to a candidate anti-Alzheimer compound;
(b) assessing said cell for one or more phenotypic hallmark of Alzheimer's disease;
(c) selecting a candidate compound capable of inhibiting and/or reducing said one or one or more phenotypic hallmark of Alzheimer's disease;
(d) modifying the chemical structure of candidate compound of step (c) to obtain a modified compound with an improved anti-Alzheimer activity (e.g. one or more of an improved biological activity, stability, absorption, blood brain barrier transfer, etc.).

In one embodiment the method further comprises repeating steps (a) to (c) with the modified compound of step (d), and optionally also repeating step (d). Therefore, in preferred embodiments, the compound is improved upon each iteration of the method and the steps (a) to (c) (and optionally step (d)) are repeated until a compound having the desired anti-Alzheimer activity is obtained.

Yet, an additional aspect of the invention concerns a method for identifying a potential biological target of a drug for use against dementia-related neurological diseases, including Alzheimer's disease and more particularly sAD. According to one particular aspect, the invention concerns a method for identifying a potential biological target of an anti-Alzheimer drug, the method comprising the steps of:
(a) making a quantitative comparative proteomic analysis or a genome wide expression comparative analysis of a BMI1-deficient primate cortical neuronal cells as defined herein with wild-type cortical neurons;
(b) identifying genes or proteins for which expression or post-translational modification is different (i.e. BMI1-deficient primate cortical neuronal cells vs wild-type cortical neurons);
wherein a gene or protein identified at step (b) is a potential biological target of an anti-Alzheimer drug.

The method may further comprises additional steps, for instance:
(c) exposing the BMI1-deficient primate cortical neuronal cells and/or the wild-type cortical neurons to an anti-Alzheimer drug;
(d) making a comparative quantitative proteomic analysis or comparative gene expression analysis of the exposed cell and/or cortical neurons with the quantitative proteomic analysis of step (a).

### Diagnostic Applications

Identification of the genetic etiology of dementia in humans (e.g. Alzheimer's disease, frontotemporal dementia and dementia with Lewy Bodies), and more particularly the genetic etiology of Alzheimer's disease and sAD, opens new avenues in the diagnostic of the diseases.

Therefore, another aspect on the present invention concerns diagnostic methods for dementia-related neurological diseases, including Alzheimer's disease and more particularly sAD in subject.

As used herein, the term “subject” includes living organisms in which Alzheimer's disease may occur. The term “subject” includes animals (e.g., mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as avian (e.g. chickens, ducks, Peking ducks, geese), and transgenic species thereof. Preferably, the subject is a human or a non-human primate (e.g., chimpanzee, monkey, macaque, gorilla). More preferably, the subject is a human.

According to one particular aspect, the invention relates to a method for diagnosing Alzheimer's disease in a subject. In one embodiment the method comprises assessing BMI1 activity in brain cells of the subject, wherein a reduced or absence of BMI1 activity is indicative of Alzheimer's disease. In embodiments the brain cells are cells from the hippocampus, cells from the frontal cortex, cells from the entorhinal cortex and/or mixtures thereof.

An additional related aspect of the invention concerns a method of diagnosing sporadic Alzheimer's disease in a human subject. In one embodiment, the method comprises detecting epigenetic BMI1 silencing in the hippocampus, in the frontal cortex and/or in the entorhinal cortex of the subject and/or in cells isolated therefrom.

Any suitable method or technique may be use for assessing BMI1 activity and/or BMI1 silencing in brain and/or in isolated brain cells of a subject. For instance, the assessing may comprise administering to the subject a radioactive and/or fluorescent blood brain permeable molecule having affinity to BMI1. Examples of molecules having affinity to BMI1 are PTC Therapeutics' BMI1 inhibitors PTC-596 and PTC-209 (see hereinbefore). The assessing of BMI1 activity and/or BMI1 silencing in the brain cells may comprises using a positron emission tomography (PET) brain scan, e.g. for detecting the radioactive and/or fluorescent blood brain permeable molecule having affinity to BMI1.

Alternatively, the assessing of BMI1 activity and/or BMI1 silencing in the brain cells may comprise obtaining a tissue biopsy from a living-subject or it may be carried out post-mortem.

The assessing of BMI1 activity and/or BMI1 silencing in the brain cells may also comprises detecting an expression level of BMI1 and/or detecting BMI1 biological activity. For instance, detecting the expression level of BMI1, detecting BMI1 biological activity and/or detecting BMI1 silencing may involve methods and techniques such as qPCR, DNA micro-array, RNA-Sequencing, ELISA, immunohistochemistry, Western blotting and/or a combination thereof.

### EXAMPLES

### Example 1: Differentiation of Human Embryonic Stem Cells into Cortical Neurons

Human embryonic stem cells were differentiated into cortical neurons. The differentiation protocol was based on a previous study (Espuny-Camacho, I., et al., Pyramidal neurons derived from human pluripotent stem cells integrate efficiently into mouse brain circuits in vivo. Neuron 77, 440-456, (2013)). However, the Noggin agonist LDN193189 was used to reduce recombinant Noggin concentration. The H9 (WiCell™) and HUES9 (Harvard Stem Cell Institute) cell lines were dissociated using Accutase™ (Innovative Cell Technology #AT-104) and platted on growth factor reduced Matrigel™ (Corning #356231) in PeproGrow™ hES cell media (PeproTech #BM-hESC) supplemented with ROCK™ inhibitor (Y-27632; 10 μM, Cayman Chemical #10005583). Upon 70% of confluency, the media was changed to DDM supplemented with B27 (1× final), Noggin (10 ng/ml, PeproTech #120-10C) and LDN193189 (0.5 μM; Sigma #SML0559). The medium was changed every day. After 16 days of differentiation, the medium was changed to DDM/B27 and replenished every day. At day 24, neural progenitors were manually detached from the plate and mixed with lentiviral supernatant for 30 min at 37° C. Infected neural progenitors were subsequently mixed in DDM/B27 supplemented with ROCK™ inhibitor (Y-27632; 10 μM) and platted on growth factor reduced Matrigel™ coated plates or chamber slides (LabTek™ #154534). Five days after the dissociation/infection, half of the medium was changed for Neurobasal™ A media supplemented with B27 (1× final) and changed again every three days. For 3D cultures, neural progenitors at day 24 were mixed with ice-cold growth factor reduced Matrigel™ (1:20 dilution) and plated onto 8-well chamber slides. After 30 min of incubation at 37° C., fresh Neurobasal™ A/B27 were added.

Sequence-specific oligonucleotides stretch shRNA designed to target the BMI-1 ORF (accession No.: BC011652): were synthetized. Oligo#1 (nt 1061-1081) 5′-CCTAATACT TTCCAGATTGAT-3′ (SEQ ID NO: 3) and oligoScramble (nt 573-591) 5′-GGTACTTCATTGATGCCAC-3′ (SEQ ID NO: 4) were used in this study. These sequences are followed by the loop sequence (TTCAAGAGA) (SEQ ID NO: 5) and finally the reverse complements of the targeting sequences. The double stranded shRNA sequences were cloned downstream of the H1P promoter of the H1 P-UbqC-HygroEGFP plasmid using Age1, SmaI, and XbaI cloning sites. The shRNA-expressing lentiviral plasmids were cotransfected with plasmids pCMVdR8.9 and pHCMV-G into 293FT packaging cells using Lipofectamin™ (Invitrogen) according to the manufacturers instructions. Viral containing media were collected, filtered, and concentrated by ultracentrifugation. Viral titers were measured by serial dilution on 293T cells followed by microscopic analysis 48 hr later. For viral transduction, lentiviral vectors were added to dissociated cells prior to plating. Hygromycin selection (150 μg/ml) was added 48 h later.

Similar results may be obtained when using human induced pluripotent stem (iPS) cells to generate cortical neurons. Similar results may be obtained also when using different shRNA sequences against human BMI1 cDNA for BMI1 knockdown.

### Example 2: Mice Hemizygous for Bmi1 Develop AD Pathology with Age

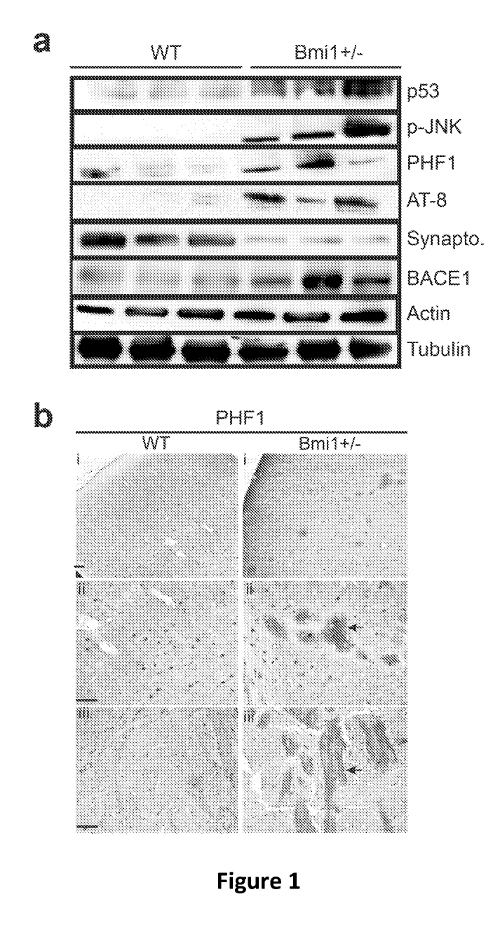

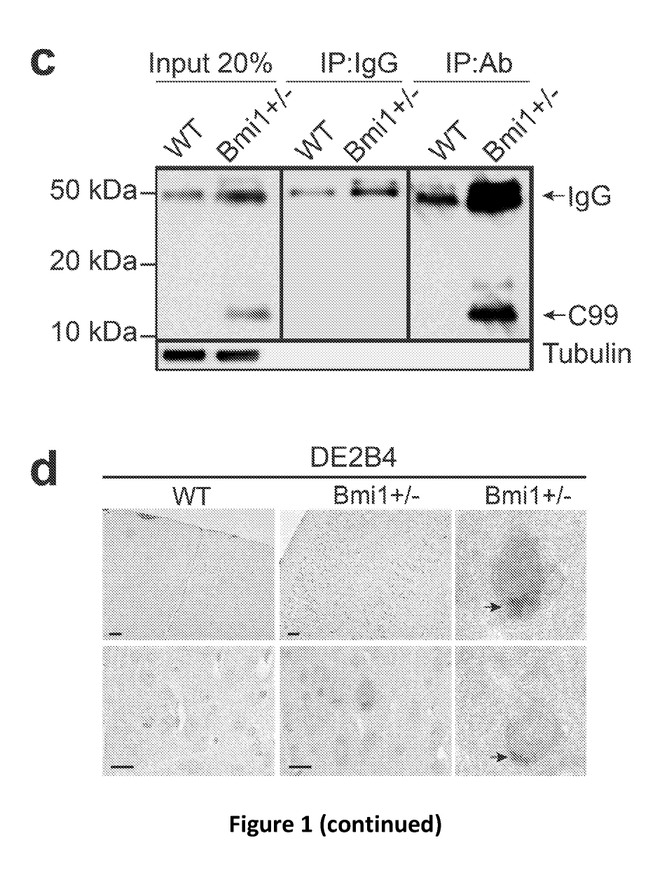

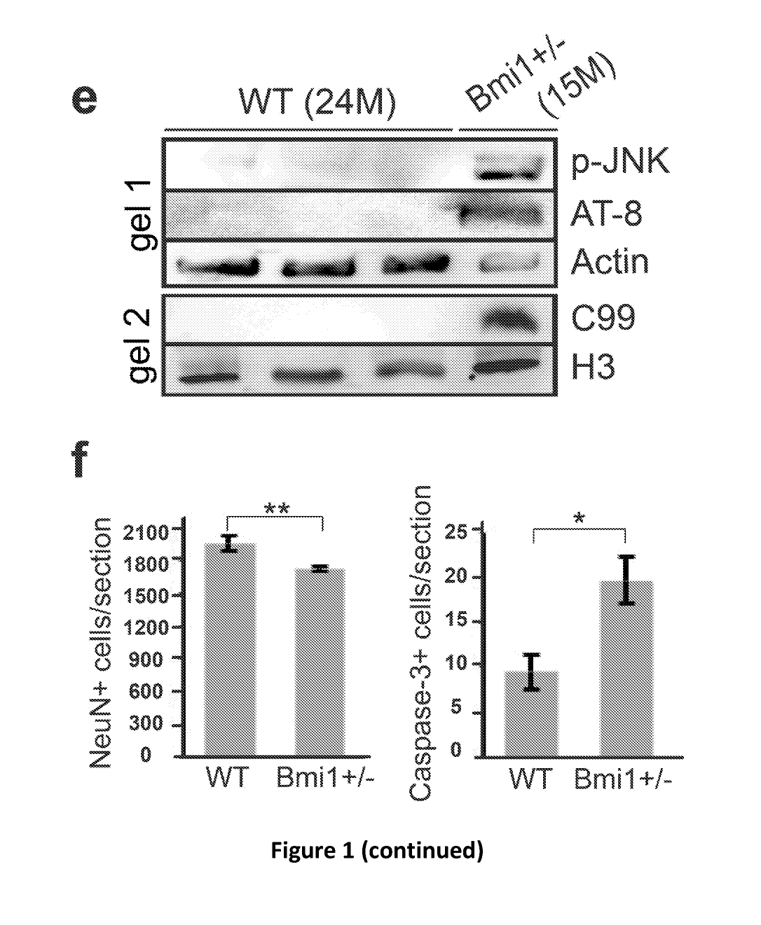

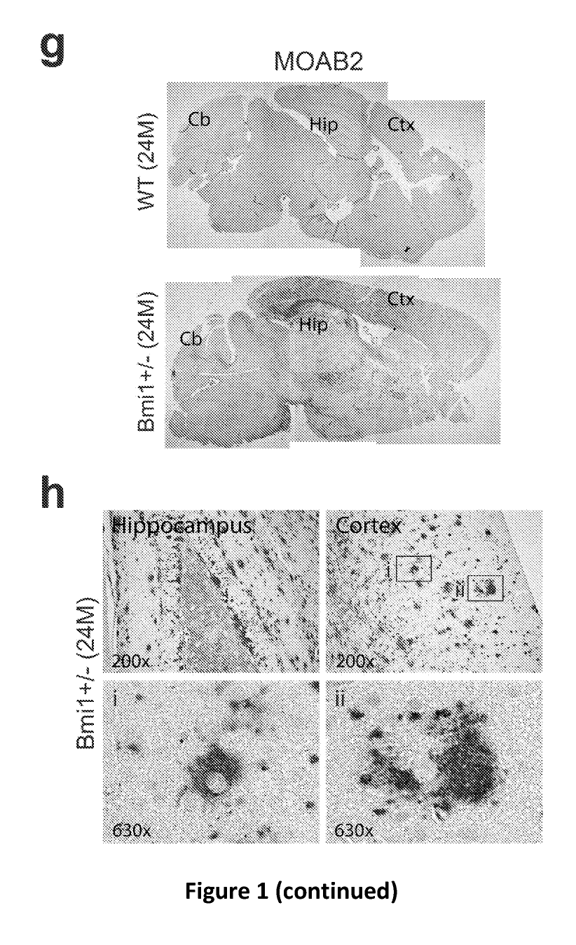

The AD-like brain (cortex) pathology of 15-24 month old Bmi1+/− mice compared to WT littermates. FIG. 1 reveals that p-Tau, C99 fragment and amyloid plaques accumulation, neuronal loss and synaptic atrophy in Bmi1+/− mice. These are recognized hallmarks of AD.

### Example 3: BMI1 Inactivation in Human Neurons Induces a Gene-Expression Signature Related to AD

To test if BMI1 deficiency in human neurons could result in a similar neuropathology, we inhibited BMI1 activity in cortical neurons produced from the differentiation of human embryonic stem (hES) cells {Espuny-Camacho, 2013}. After 24 days of neural induction, progenitors were dissociated and infected with a lentivirus expressing a small hairpin RNA (shRNA) with a scramble sequence (shCTL+Hygro-/GFP) or an shRNA directed against BMI1 (shBMI1+Hygro/GFP), prior to differentiation in post-mitotic neurons (FIG. 2A-C) {Abdouh, 2009}. After 14 days, the majority of cells were positive for the anterior neural/cortical marker FOXG1, the pan-neural markers b-III-tubulin, MAP2 and NeuN, and the glutamatergic and GABAnergic neural markers vGLUT1 and GABA, respectively (FIG. 2D-G). Morphometric analyses using the PMI index revealed that ˜60% of bIII-tubulin neurons were cortical pyramidal neurons (FIG. 2H) {Espuny-Camacho, 2013; Hand, 2005}. The proportion of vGLUT1 and GABA positive neurons was comparable with that reported for purified neuronal cultures {Israel, 2012} (FIG. 2G) {de Silva, 2003}. Axonal swellings are present at the earliest stages of AD {Stokin, 2005}. Notably, dystrophic neurites and axonal swelling could be readily observed 7-15 days after BMI1-knockdown (FIG. 2D). We also observed GABA-positive neurons with dystrophic neurites and axonal swellings, and disrupted vGLUT1 or bIII-tubulin labeling along axonal segments (FIG. 2E-F). Comparative gene expression profile analysis confirmed efficient neuronal differentiation of the cultures (FIG. 2I). Since BMI1 operates as a transcriptional repressor, we extracted most up-regulated genes in shBMl1 neurons when compared to controls. Unbiased gene ontology analysis revealed that these were predominantly associated with AD-associated signaling pathways, namely Cadherin, WNT and AD-presenilin {Jackson, 2002; Ando, 2011; Fraser, 2001}, as well as Huntington disease and nicotinic acetylcholine receptor pathways (FIG. 2J). We annotated the top 14 most significant up-regulated genes. Transthyretin (TTR) inhibits Ab42 aggregation and behaves as a neuronal stress-response protein {Wang, 2014}. DNAJA4 (also known as HSP40) is a molecular chaperone that prevents protein misfolding and amyloid aggregation {Torrente, 2013}. UBR1 is an ubiquitin ligase involved in recognition and targeting of normal and misfolded proteins for poly-ubiquitylation and proteosomal degradation {Kim, 2014}. These results revealed that BMI1 inactivation resulted in a gene-expression signature highly related to AD, which also included early activation of cellular response against protein misfolding.

Overall, these results reveal that upon loss of BMI1, human cortical neurons display several anomalies such as axonal swelling, axonal segment breaks, and a gene-expression signature highly related Cadherin/Wnt and Alzheimer's disease/Presenilin signaling.

### Example 4: BMI1 Deficiency in Human Cortical Neurons Recapitulates AD Pathological Hallmarks—Accumulation of p-Tau and Amyloid

AD-associated p-Tau can sequester normal Tau and lead to tangle formation and microtubule disassembly {Alonso, 1996}. Using PHF1 and S422, we observed p-Tau immunoreactivity in shBMl1 neurons (FIG. 3A-C), and this in both axonal and dendritic compartments (not shown). Consistently, axonal segments where vGLUT1 or bIII-tubulin distribution was perturbed also presented focal accumulations of p-Tau (FIG. 3B). Diffuse PHF1 immunoreactivity was also observed within axonal swellings (FIG. 3B-inset). In contrast, PHF1 weakly decorated the axons of some control neurons (FIG. 3A-C). The frequency of apoptotic nuclei was also significantly higher in shBMl1 neurons (FIG. 3D-E). Using antibodies against Ab42, we observed robust immunoreactivity in the soma and neurites of shBMI1 neurons when compared to controls (FIG. 3F), and amyloid immunoreactivity was highly reduced upon exposure to BACE inhibitor (BSI) (FIG. 3G). Similar observations were made using NU1, which recognizes oligomeric amyloid (FIG. 3G) {Lambert, 2007; Kondo, 2013}. Using ELISA, Ab42 was detected in control neuronal extracts and culture media, but the relative amyloid concentration was increased in the corresponding BMI1-knockdown samples at day 7 and 14, and amyloid concentration was normalized upon treatment with BSI (FIG. 3H). Amyloid immunoreactivity was not observed in non-neuronal cells knockdown for BMI1 (Data not shown). The neuropathology was also observed when using a distinct shRNA sequence against BMI1n (Data not shown), or when introducing the shBMl1 DNA construct in Ki67-, Nestin- and PCNA-negative post-mitotic neurons (Data not shown).

### Example 5: BMI1 Deficiency In Human Cortical Neurons Recapitulates AD Pathological Hallmarks—Formation of p-Tau Tangles and Amyloid Plagues Formation

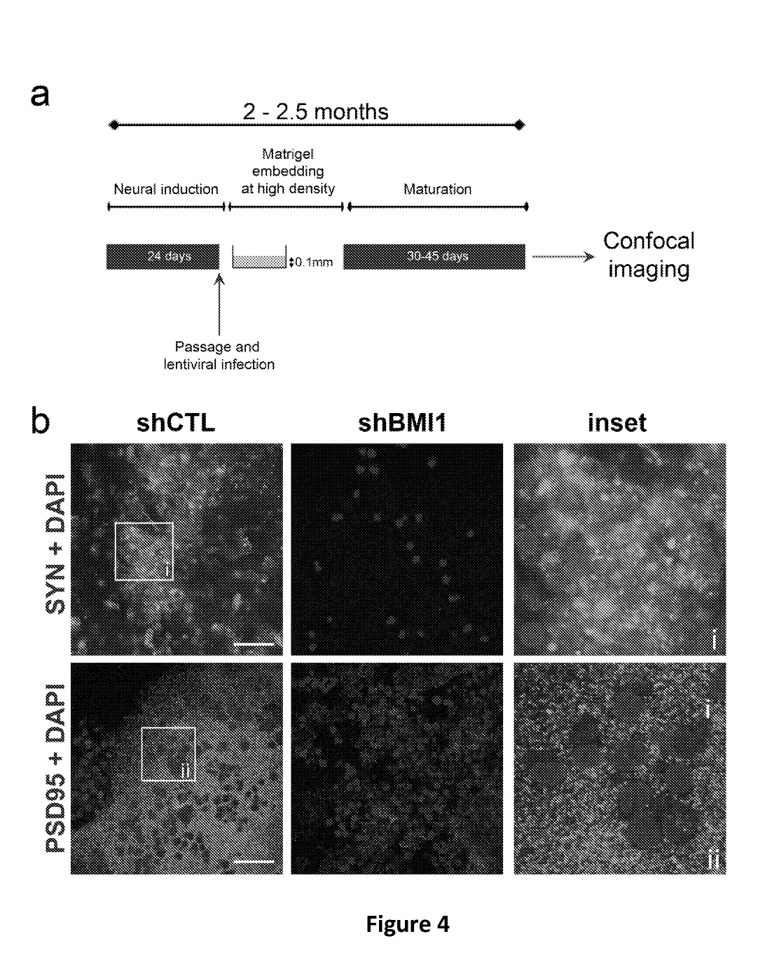

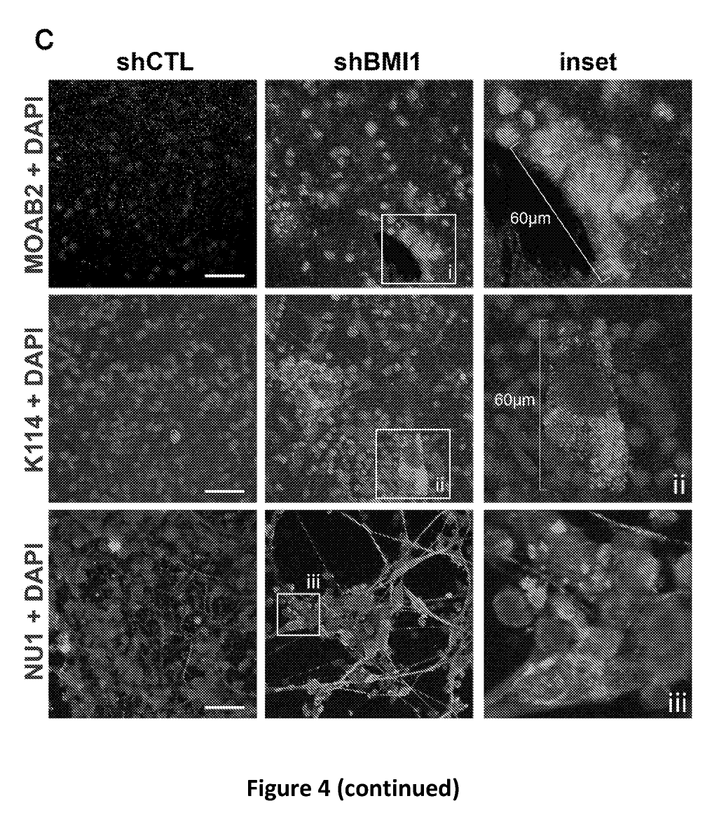

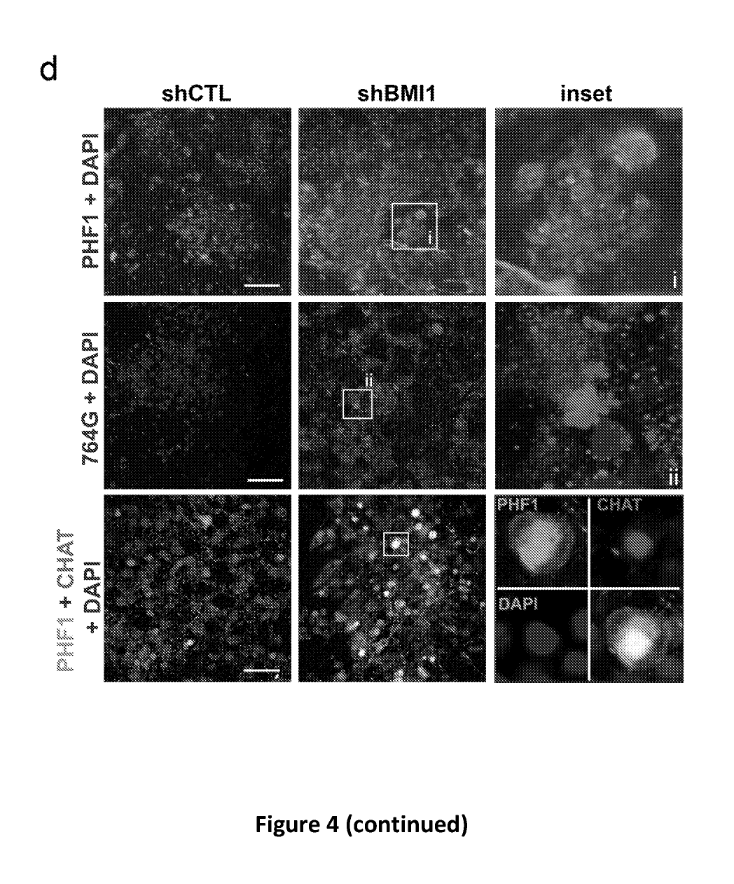

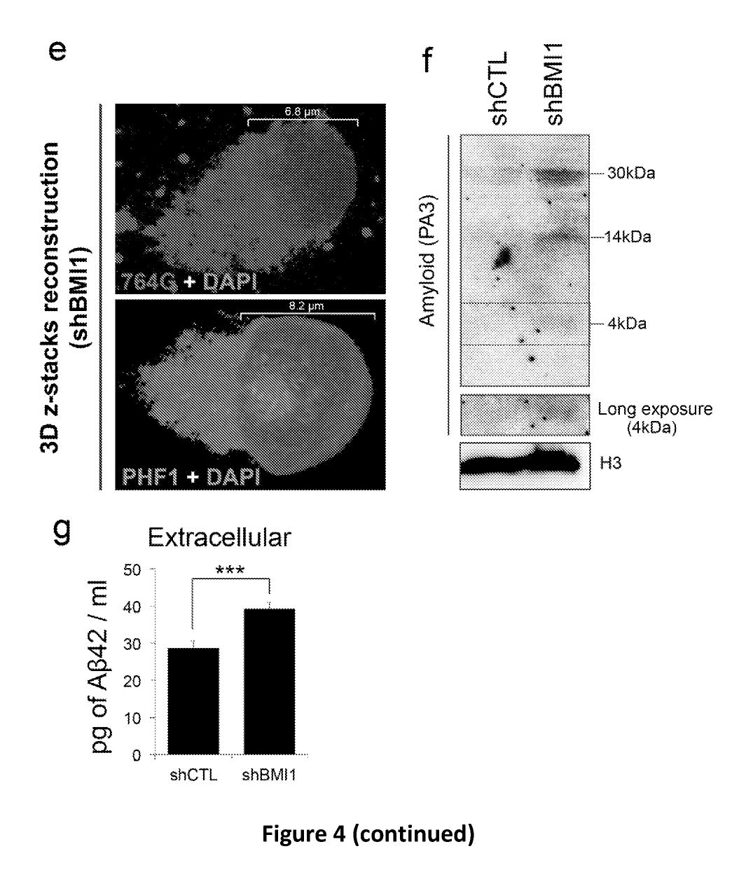

It was shown that FAD genes-expressing neuronal cultures grown in 3D could generate amyloid plaques and Tau-tangles {Choi, 2014}. By adapting this method (FIG. 4A), we found that in contrast to control cultures, which underwent pre-(synaptophysin) and post-(PSD-95) synaptic maturation, BMI1 deficient neuronal cultures failed to express significant amounts of synaptophysin and PSD-95 (FIG. 4B). BMI1 knockdown also resulted in the formation of extra-cellular amyloid plaques, as revealed using specific antibodies or the auto-fluorescent compound K114, which binds to amyloid fibrills and plaques (FIG. 4C). These conditions also resulted in the formation of diffuse extra-cellular p-Tau deposits and large p-Tau tangles located in the neuronal soma (FIG. 4D-E). The presence of p-Tau aggregates was observed in degenerating ChAT-positive cholinergic neurons, a neuronal cell type affected in AD (FIG. 4D) {Baskin, 1999}. BMI1 deficient cultures were also characterized by the accumulation of amyloid species of ˜30 kDa, ˜14 kDa and ˜4 kDa as revealed by immuno-blot using cellular extracts, or by ELISA using the cell culture supernatant (FIG. 4F-G). This revealed that BMI1 inactivation in human neurons recapitulated AD pathological hallmarks.

### Example 6: BMI1 Deficiency Results in Pathological Activation of MAPT, GSK3b and p53

To get insight of the disease mechanism, we analyzed the expression of AD-related genes in neuronal cultures. CDKN2A(p16) and CDKN1A(p21) were used as control genes up-regulated upon BMI1 deficiency {Abdouh, 2009; Chatoo, 2009}. Only MAPT (encoding for Tau) and LRP2 were increased upon BMI1 deficiency, while APP expression was reduced (Data not shown). Since MAPT duplication is pathogenic {Rovelet-Lecrux, 2010}, we investigated possible regulation of MAPT by BMI1. Accordingly, MAPT expression was reduced upon BMI1 over-expression in naive neurons (Data not shown), and BMI1 was enriched on several portions of the MAPT locus—including the promoter region—in control neurons, as measured by Chromatin Immuno-Precipitation (ChIP) using a specific BMI1 antibody (Data not shown), suggesting direct transcriptional repression. BMI1 can modulate the DNA damage response (DDR) {Facchino, 2010; Ismail, 2010}. BMI1/RING1a/b can also promote p53 degradation through transcriptional repression of p14^{ARF} (the second CDKN2A-encoded transcript), releasing the activity of the MDM2 ubiquitin ligase {Sherr, 2001}, and through interaction with p53, leads to monoubiquitination of p53 {Calao, 2013}. Considering this, we surmised that transcription-independent mechanisms could explain some of the observed pathologies. Likewise, GSK3b can phosphorylate Tau at Ser396/404 (as detected with PHF1), and GSK3b expression and activity are regulated by translational and post-translational mechanisms {Medina, 2014}.

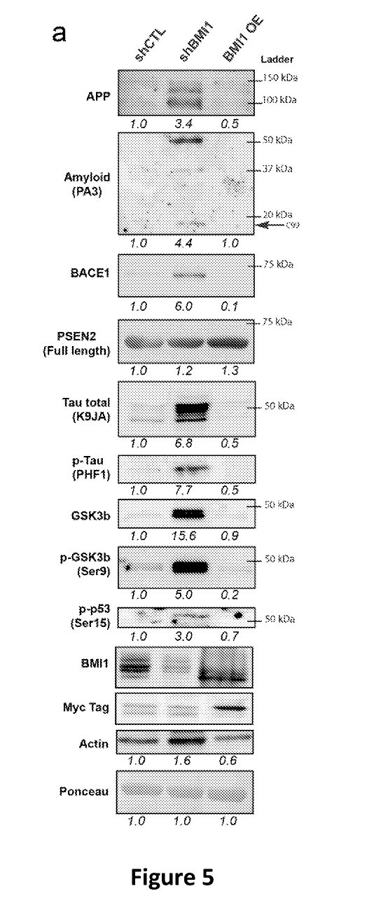

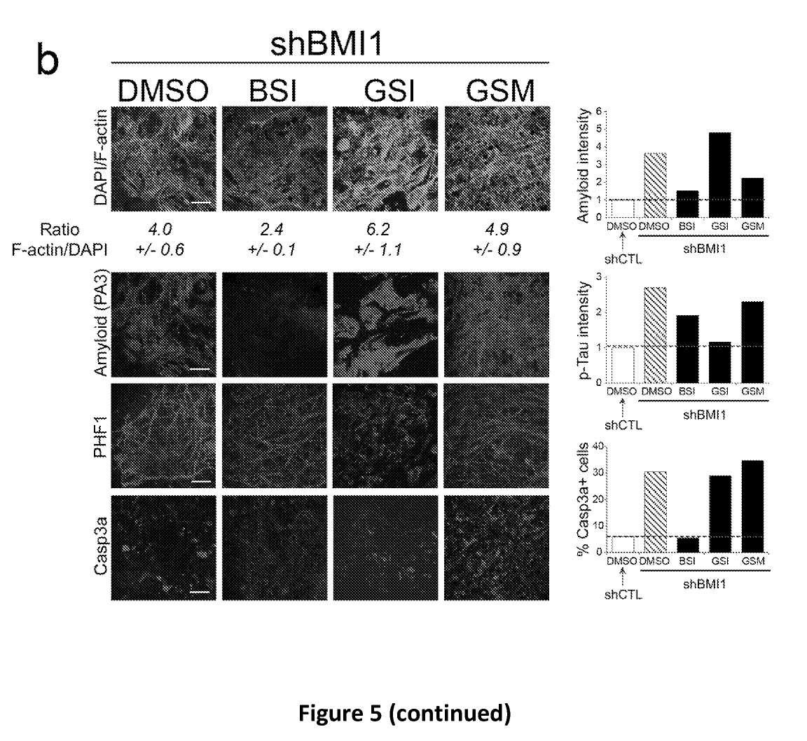

By immunoblot, we detected a dramatic increase in Tau, p-Tau, GSK3b, p-GSK3b (Ser9) and p-p53 (Ser15) levels in BMI1-deficient neurons (FIG. 5A). Conversely, BMI1 over-expression in naive neurons could reduce baseline levels of Tau, p-Tau, GSK3b, p-GSK3b and p-p53 (FIG. 5A). BMI1 knockdown also resulted in elevated APP, BACE1 and b-Actin expression when compared to controls, while BMI1 over-expression resulted in the opposite trend (FIG. 5A). Accordingly, we observed accumulation of C99 and of two larger amyloid-related fragments in BMI1 knockdown neurons (FIG. 5A). Notably, the elevation in monomeric b-Actin levels correlated with F-Actin bundling in BMI1 knockdown neurons, as visualized by IF using phalloidin (FIG. 5B). F-Actin bundling is present in AD and tauopathies and is largely dependent on cross-linking activities of Tau with F-Actin and Tubulin dimers {Elie, 2015}. We next analyzed the impact of BSI, g-secretase inhibitor (GSI) and g-secretase modulator (GSM) on BMI1-deficient neurons {Choi, 2014}. BSI reduced apoptosis and amyloid immuno-reactivity, but only partially that of p-Tau and F-Actin (FIG. 5B), suggesting that C99 production and toxicity could be partly uncoupled from p-Tau accumulation. On the other hand, GSI and GSM did not improve most pathological parameters (FIG. 5B). While GSI reduced p-Tau intensity, the re-localization of p-Tau to the neuronal soma suggested tangle formation (FIG. 5B). Furthermore, amyloid immuno-reactivity reached saturation levels upon GSI treatment, possibly owing to C99 accumulation (FIG. 5B) {Moore, 2015}.

### Example 7: BMI1 is Silenced in AD Brains and Enriched at the MAPT Locus in Normal Brains

To investigate BMI1 expression in human brains, we established the best normalization gene to be used when comparing the hippocampus of AD and control samples. For the same amount of total RNA, we found that GAPDH and FOXG1 showed no variation in Ct value between all samples as compared to ASCL1, NEUROD1, ACTB, NGN2, NeuN and RPL13, which showed the strongest variations (Data not shown). When normalized to GAPDH, FOXG1, ASCL1 or NEUROD1, BMI1 expression was significantly reduced in AD samples, and comparable results were obtained when using distinct primer-pairs covering the BMI1 cDNA (FIG. 6A-C). In contrast, expression of EZH2, another Polycomb group gene, was not altered in AD samples (FIG. 6D). BMI1 expression was also reduced in the frontal cortex of AD samples, but not of FAD samples (Data not shown). On frontal cortex sections, BMI1 expression was observed in NeuN-positive pyramidal neurons in control samples and was reduced in AD samples (FIG. 6E). Consistently with the gene expression results, BMI1 protein expression (using the F6 antibody) was not affected in the hippocampus of FAD patients (n=4, median age of 61 year) when compared to controls (n=3, median age of 59 years) (FIG. 6F and FIG. 6K). In contrast, BMI1 was significantly reduced in the hippocampus of AD patients (n=5, median age of 82 years) when compared to controls (n=3, median age of 87 years) (FIG. 6F and FIG. 6J-K). In the frontal cortex, we observed the presence of a BMI1 triplet representing different phosphorylated forms of BMI1 in control samples (n=6, median age of 87 years), (FIG. 6G) {Voncken, 1999}. In contrast, BMI1 expression was near absent in AD samples (n=6, median age of 87 years) (FIG. 6G and FIG. 6K). Histone H2A is the main target of BMI1/RING1a/b activity {Buchwald, 2006; Li, 2006}. H2A^{ub} levels were significantly reduced in AD brains but not in FAD brains (FIGS. 6F-G and 6K).

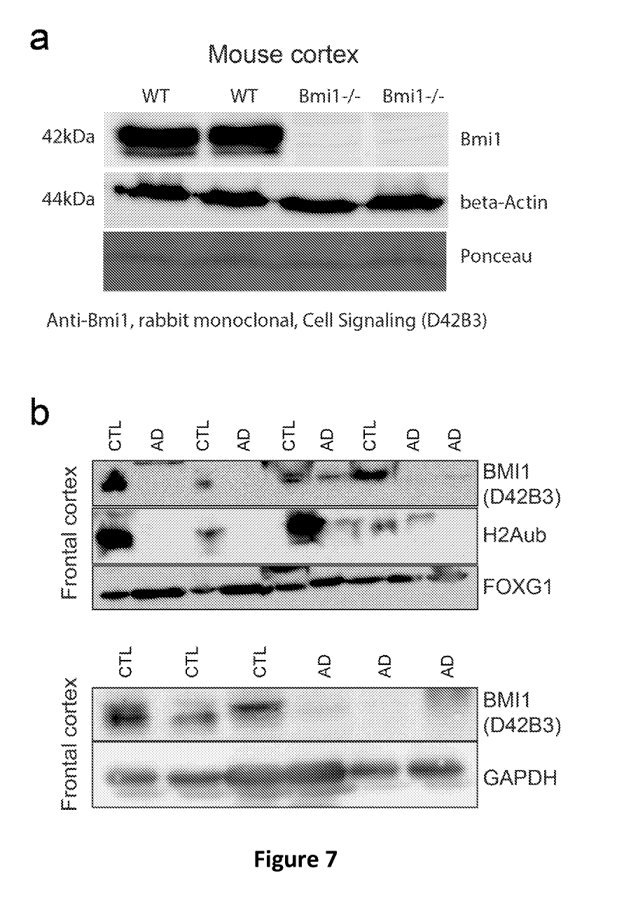

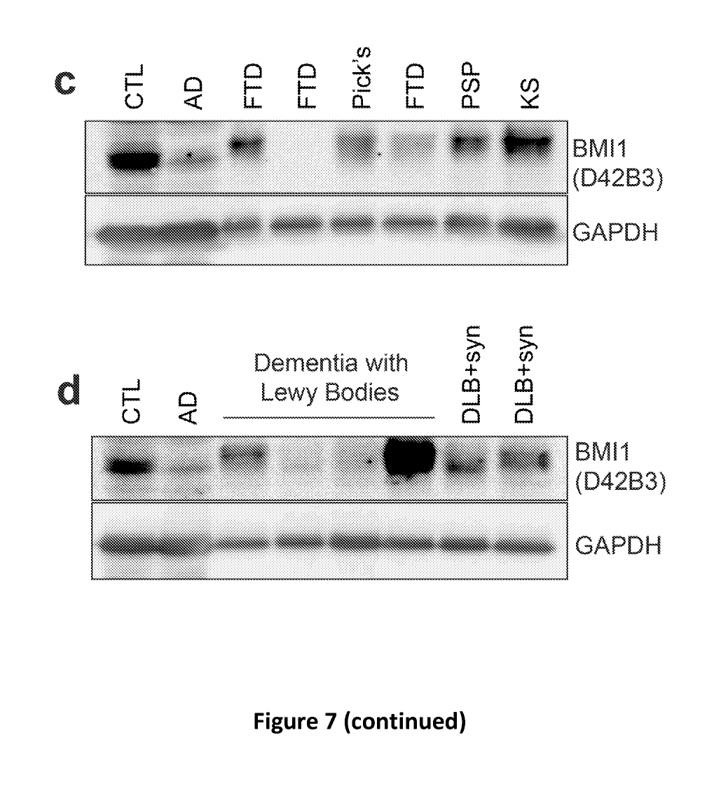

Comparable results were obtained when using a second specific BMI1 antibody (D42B3) (FIG. 7A-B). BMI1 levels were not significantly reduced in frontal cortex samples from patients with other dementias, with few exceptions (FIG. 6H and FIG. 7A-B). Using a third BMI1 antibody (1.T.21), we could confirm that BMI1 and H2A^{ub} were enriched at the MAPT and INK4A loci in the non-demented human cortex and depleted in the AD cortex (FIG. 6I).

### Example 8: BMI1 Silencing Causes Heterochromatin Anomalies, a Hallmark of Neurodegeneration

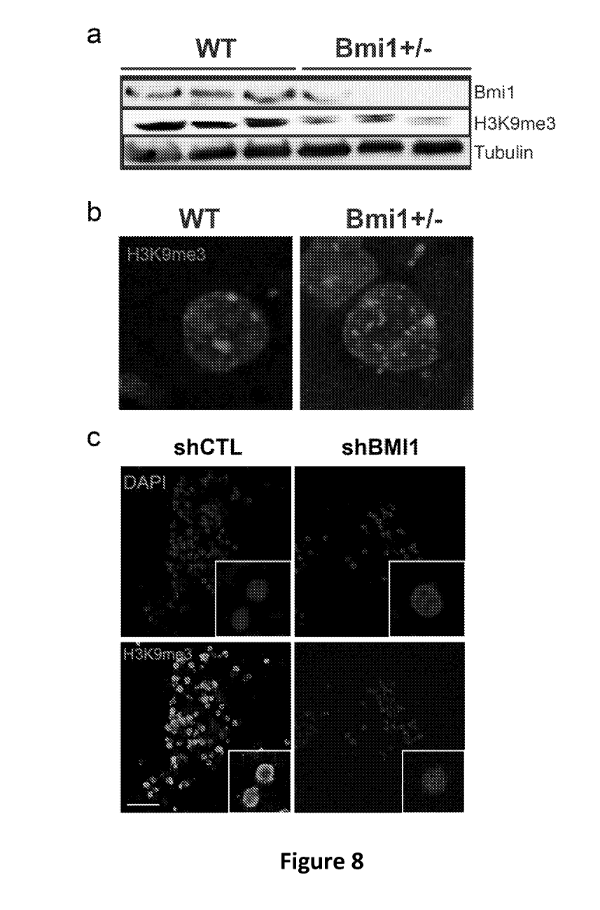

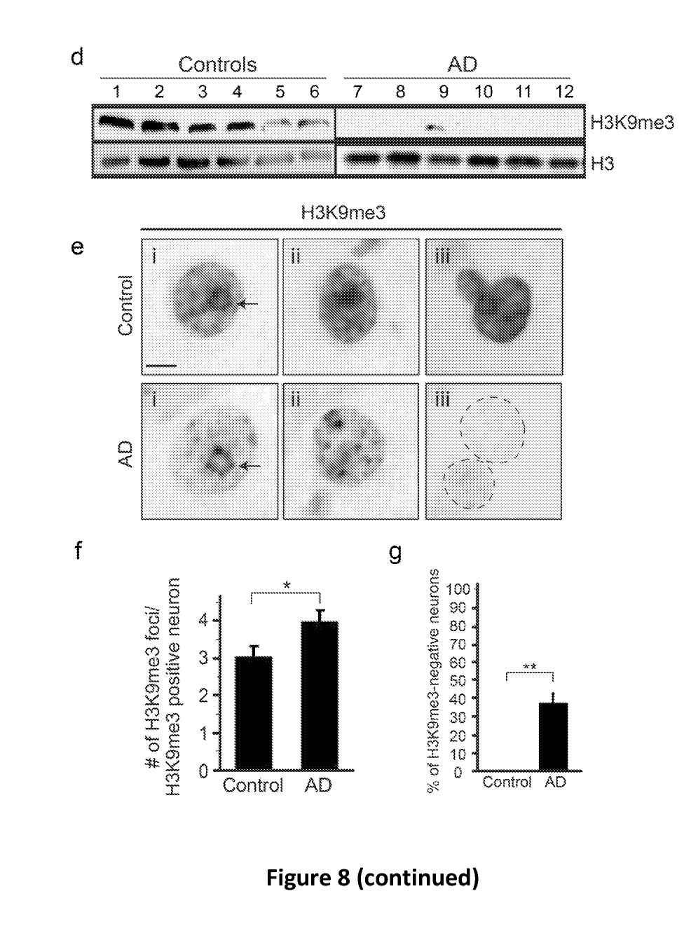

To determine whether BMI1 loss could induce heterochromatin anomalies as seen in neurodegenerative disorders, we analyzed old (24-months) Bmi1^{+/-} mice and hES cells-derived cortical neurons knocked-down for BMI1 (shBMI1). The tri-methylation of histone H3 at the Lysine 9 (H3K9me3) is a marker of heterochromatin. H3K9me3 levels were decreased in the cortex of Bmi1^{+/-} mice as compared to WT littermates (FIG. 8A). Notably, H3K9me3 foci were dissociated from the condensed chromatin (FIG. 8B). Similar results were observed in shBMl1 human cortical neurons compared to shCTL neurons (FIG. 8C). Likewise, sAD human cortical samples also have reduced levels of H3K9me3 (FIG. 8D-G).

### Example 9: BMI1 Inactivation in Cortical Neurons Using CRISPR/Cas9

To further validate our findings, we used a genetic approach, the Clustered Regularly Interspaced Short Palindromic Repeats (CRISPR)/CRISPR-associated (Cas) system (CRISPR/Cas), to introduce a bi-allelic null deletion at the BMI1 locus (Mali et al., 2013). BMI1 inactivation using CRISPR/Cas9 was carried out by the polymeric delivery of a Cas9-expressing plasmid (Dharmacon #CAS10140), a synthetic guide RNA (sgRNA) Scramble (Dharmacon #U-007501) or complementary to BMI1 (Target: AACGTGTATTGTTCGTTACC; SEQ ID NO: 6) and a synthetic trans-activating crRNA (Dharmacon #U-002005) using Mirus TransIT-X2™ (Cat #M1R6003) according to manufacturer instructions.

Briefly, a plasmid encoding for the Cas9 nuclease together with a control guide RNA (sgRNA-Ctrl) or a guide RNA targeting BMI1 exon 1 (sgRNA-BMI1) were transfected in 30 days old naive human post-mitotic neurons using a polymeric delivery agent (FIG. 9A). Ten days later, neurons were analyzed by genomic PCR. This revealed the presence of a ˜400 bp deletion in exon I of BMI1) (BMI1^{KO}) only in neurons exposed to sgRNA-BMI1 (FIG. 9B). Notably, loss of BMI1 activity was observed in greater than 90% of cells in BMI1^{KO} cultures, as revealed using antibodies against BMI1 and H2A^{ub} (FIG. 9C-D). In contrast to other methods, this approach did not require viral infection, drug-selection and/or cell sorting (Rubio et al., 2016). Notably, BMI1^{KO} neuronal cultures were characterized by the presence of piknotic nuclei and the accumulation of p-Tau and beta-amyloid (FIG. 9E-F). p-Tau immuno-reactivity was furthermore visualized at the single cell level in H2A^{ub}-low (BMI1^{KO}) neurons, but not in H2A^{ub}-high (wild type) neurons, suggesting a cell autonomous effect (FIG. 9E). To test the role of BMI1 in synaptic maintenance, BMI1 knockout was induced in 60 days old neurons, which were analyzed 7 days later. While immuno-reactivity for PSD-95 and synaptophysin was observed in control neurons, it was strongly reduced in BMI1^{KO} neurons (FIG. 9G-H), revealing that BMI1 is required for the maintenance of synaptic integrity.

### Conclusions

The present data prove that loss-of-function of the BMI1 gene could recapitulate all Alzheimer's disease neuronal pathological features in aged mice and human cortical neurons. The inhibition of BMI1 in human neurons was accompanied by the accumulation of p-Tau along axonal segments, the formation of extra-cellular amyloid plaques and intra-neuronal p-Tau tangles, synaptic degeneration and neuronal cell death. Highly similar neuronal pathologies were found in aged Bmi1^{+/-} mice. The present molecular analyses also revealed that BMI1 could repress MAPT, suggesting that BMI1 deficiency can result in a tauopathy independently of the APP/amyloid pathway. Hence, BSI prevented apoptosis and C99/amyloid accumulation in BMI1-deficient neurons, but only partially rescued p-Tau and F-Actin levels, suggesting the presence of two parallels disease-mediating pathways. However, these pathways can be interconnected in particular contexts since inhibition of g-secretase activity, which results in C99 accumulation, can promote Tau accumulation by a yet undefined mechanism {Moore, 2015}.

As a proof of principle of the use of the present invention for the validation of drug candidates against sAD, the present inventors have tested the gamma-secretase inhibitor (GSI) that was tested and abandoned in 2014 because of its high neuro-toxicity in sAD patients {De Strooper, 2014}. The present results show that this GSI was also toxic for BMI1-knockdown human cortical neurons through an induced accumulation of the C99 fragment of APP (FIG. 5B).

The present inventors have found also that BMI1 (also called PCGF4) is preferentially silenced in sAD brains and that BMI1 deficiency results in AD in mice and cultured human cortical neurons. This indicates that epigenetic BMI1 silencing is the likely cause of sAD. The results obtained further confirmed that BMI1 is required for the maintenance of synaptic integrity. Accordingly, the present invention may help to fill the immense knowledge-gap and therapeutic vacuum for sAD associated with the lack of a valid experimental model of sAD.

### REFERENCES

Abdouh, M., Facchino, S., Chatoo, W., Balasingam, V., Ferreira, J., and Bernier, G. (2009). BMI1 sustains human glioblastoma multiforme stem cell renewal. J. Neurosci. 29, 8884-8896.

Abdouh, M., et al. (2012). Bmi1 is down-regulated in the aging brain and displays antioxidant and protective activities in neurons. PLoS One 7(2): e31870.

Alonso, A. C., Grundke-lqbal, I., and Iqbal, K. (1996). Alzheimer's disease hyperphosphorylated tau sequesters normal tau into tangles of filaments and disassembles microtubules. Nat. Med. 2, 783-787.

Ando, K., Uemura, K., Kuzuya, A., Maesako, M., Asada-Utsugi, M., Kubota, M., Aoyagi, N., Yoshioka, K., Okawa, K., Inoue, H., et al. (2011). N-cadherin regulates p38 MAPK signaling via association with JNK-associated leucine zipper protein: implications for neurodegeneration in Alzheimer disease. J. Biol. Chem. 286, 7619-7628.

Baskin, D. S., Browning, J. L., Pirozzolo, F. J., Korporaal, S., Baskin, J. A., and Appel, S. H. (1999). Brain choline acetyltransferase and mental function in Alzheimer disease. Arch. Neurol. 56, 1121-1123.

Blennow, K., de Leon, M. J., and Zetterberg, H. (2006). Alzheimer's disease. Lancet 368, 387-403.

Buchwald, G., van der Stoop, P., Weichenrieder, O., Perrakis, A., van Lohuizen, M., and Sixma, T. K. (2006). Structure and E3-ligase activity of the Ring-Ring complex of polycomb proteins Bmi1 and Ring1 b. EMBO J. 25, 2465-2474.

Calao, M., Sekyere, E. O., Cui, H. J., Cheung, B. B., Thomas, W. D., Keating, J., Chen, J. B., Raif, A., Jankowski, K., Davies, N. P., et al. (2013). Direct effects of Bmi1 on p53 protein stability inactivates oncoprotein stress responses in embryonal cancer precursor cells at tumor initiation. Oncogene 32, 3616-3626.

Chatoo, W., Abdouh, M., David, J., Champagne, M. P., Ferreira, J., Rodier, F., and Bernier, G. (2009). The polycomb group gene Bmi1 regulates antioxidant defenses in neurons by repressing p53 pro-oxidant activity. J. Neurosci. 29, 529-542.

Choi, S. H., Kim, Y. H., Hebisch, M., Sliwinski, C., Lee, S., D'Avanzo, C., Chen, H., Hooli, B., Asselin, C., Muffat, J., et al. (2014). A three-dimensional human neural cell culture model of Alzheimer's disease. Nature 515, 274-278.

de Silva, R., Lashley, T., Gibb, G., Hanger, D., Hope, A., Reid, A., Bandopadhyay, R., Utton, M., Strand, C., Jowett, T., et al. (2003). Pathological inclusion bodies in tauopathies contain distinct complements of tau with three or four microtubule-binding repeat domains as demonstrated by new specific monoclonal antibodies. Neuropathol. Appl. Neurobiol. 29, 288-302.

De Strooper, B., and Chavez Gutierrez, L. (2014). Learning by Failing: Ideas and Concepts to Tackle gamma-Secretases in Alzheimer's Disease and Beyond. Annu. Rev. Pharmacol. Toxicol. 55, 419-437.

Elie, A., Prezel, E., Guerin, C., Denarier, E., Ramirez-Rios, S., Serre, L., Andrieux, A., Fourest-Lieuvin, A., Blanchoin, L., and Arnal, I. (2015). Tau co-organizes dynamic microtubule and actin networks. Sci Rep 5, 9964.

Espuny-Camacho, I., Michelsen, K. A., Gall, D., Linaro, D., Hasche, A., Bonnefont, J., Bali, C., Orduz, D., Bilheu, A., Herpoel, A., et al. (2013). Pyramidal neurons derived from human pluripotent stem cells integrate efficiently into mouse brain circuits in vivo. Neuron 77, 440-456.

Facchino, S., Abdouh, M., Chatoo, W., and Bernier, G. (2010). BMI1 confers radioresistance to normal and cancerous neural stem cells through recruitment of the DNA damage response machinery. J. Neurosci. 30, 10096-10111.

Fraser, P. E., Yu, G., Levesque, L., Nishimura, M., Yang, D. S., Mount, H. T., Westaway, D., and St George-Hyslop, P. H. (2001). Presenilin function: connections to Alzheimer's disease and signal transduction. Biochem. Soc. Symp., 89-100.

Hand, R., Bortone, D., Mattar, P., Nguyen, L., Heng, J. I., Guerrier, S., Boutt, E., Peters, E., Barnes, A. P., Parras, C., et al. (2005). Phosphorylation of neurogenin2 specifies the migration properties and the dendritic morphology of pyramidal neurons in the neocortex. Neuron 48, 45-62.

Hebert, L. E., Weuve, J., Scherr, P. A., and Evans, D. A. (2013). Alzheimer disease in the United States (2010-2050) estimated using the 2010 census. Neurology 80, 1778-1783.

Ismail, I. H., Andrin, C., McDonald, D., and Hendzel, M. J. (2010). BMI1-mediated histone ubiquitylation promotes DNA double-strand break repair. J. Cell Biol. 191, 45-60.

Israel, M. A., Yuan, S. H., Bardy, C., Reyna, S. M., Mu, Y., Herrera, C., Hefferan, M. P., Van Gorp, S., Nazor, K. L., Boscolo, F. S., et al. (2012). Probing sporadic and familial Alzheimer's disease using induced pluripotent stem cells. Nature 482, 216-220.

Jackson, G. R., Wiedau-Pazos, M., Sang, T. K., Wagle, N., Brown, C. A., Massachi, S., and Geschwind, D. H. (2002). Human wild-type tau interacts with wingless pathway components and produces neurofibrillary pathology in Drosophila. Neuron 34, 509-519.

Kim, H. K., Kim, R. R., Oh, J. H., Cho, H., Varshaysky, A., and Hwang, C. S. (2014). The N-terminal methionine of cellular proteins as a degradation signal. Cell 156, 158-169.

Kondo, T., Asai, M., Tsukita, K., Kutoku, Y., Ohsawa, Y., Sunada, Y., Imamura, K., Egawa, N., Yahata, N., Okita, K., et al. (2013). Modeling Alzheimer's disease with iPSCs reveals stress phenotypes associated with intracellular Abeta and differential drug responsiveness. Cell Stem Cell 12, 487-496.

Lambert, M. P., Velasco, P. T., Chang, L., Viola, K. L., Fernandez, S., Lacor, P. N., Khuon, D., Gong, Y., Bigio, E. H., Shaw, P., et al. (2007). Monoclonal antibodies that target pathological assemblies of Abeta. J. Neurochem. 100, 23-35.

Li, Z., Cao, R., Wang, M., Myers, M. P., Zhang, Y., and Xu, R. M. (2006). Structure of a Bmi-1-Ring1B polycomb group ubiquitin ligase complex. J. Biol. Chem. 281, 20643-20649.

Mali, P., Esvelt, K. M., and Church, G. M. (2013). Cas9 as a versatile tool for engineering biology. Nat Methods 10, 957-963.

Medina, M., and Avila, J. (2014). New insights into the role of glycogen synthase kinase-3 in Alzheimer's disease. Expert Opin Ther Targets 18, 69-77.

Moore, S., Evans, L. D., Andersson, T., Portelius, E., Smith, J., Dias, T. B., Saurat, N., McGlade, A., Kirwan, P., Blennow, K., et al. (2015). APP metabolism regulates tau proteostasis in human cerebral cortex neurons. Cell Rep 11, 689-696.

Rovelet-Lecrux, A., Hannequin, D., Guillin, O., Legallic, S., Jurici, S., Wallon, D., Frebourg, T., and Campion, D. (2010). Frontotemporal dementia phenotype associated with MAPT gene duplication. J Alzheimers Dis 21, 897-902.

Rubio, A., Luoni, M., Giannelli, S. G., Radice, I., lannielli, A., Cancellieri, C., Di Berardino, C., Regalia, G., Lazzari, G., Menegon, A., Taverna, S., and Broccoli, V. (2016). Rapid and efficient CRISPR/Cas9 gene inactivation in human neurons during human pluripotent stem cell differentiation and direct reprogramming. Sci Rep 6, 37540.

Sawa, G. M., Wharton, S. B., Ince, P. G., Forster, G., Matthews, F. E., and Brayne, C. (2009). Age, neuropathology, and dementia. N. Engl. J. Med. 360, 2302-2309.

Sherr, C. J. (2001). The INK4a/ARF network in tumour suppression. Nat Rev Mol Cell Biol 2, 731-737.

Stokin, G. B., Lillo, C., Falzone, T. L., Brusch, R. G., Rockenstein, E., Mount, S. L., Raman, R., Davies, P., Masliah, E., Williams, D. S., et al. (2005). Axonopathy and transport deficits early in the pathogenesis of Alzheimer's disease. Science 307, 1282-1288.

Torrente, M. P., and Shorter, J. (2013). The metazoan protein disaggregase and amyloid depolymerase system: Hsp110, Hsp70, Hsp40, and small heat shock proteins. Prion 7, 457-463.

van der Lugt, N. M., Domen, J., Linders, K., van Roon, M., Robanus-Maandag, E., to Riele, H., van der Valk, M., Deschamps, J., Sofroniew, M., van Lohuizen, M., et al. (1994). Posterior transformation, neurological abnormalities, and severe hematopoietic defects in mice with a targeted deletion of the bmi-1 proto-oncogene. Genes Dev. 8, 757-769.

Villemagne, V. L., Burnham, S., Bourgeat, P., Brown, B., Ellis, K. A., Salvado, O., Szoeke, C., Macaulay, S. L., Martins, R., Maruff, P., et al. (2013). Amyloid beta deposition, neurodegeneration, and cognitive decline in sporadic Alzheimer's disease: a prospective cohort study. Lancet Neurol 12, 357-367.

Voncken, J. W., Schweizer, D., Aagaard, L., Sattler, L., Jantsch, M. F., and van Lohuizen, M. (1999). Chromatin-association of the Polycomb group protein BMI1 is cell cycle-regulated and correlates with its phosphorylation status. J. Cell Sci. 112 (Pt 24), 4627-4639.

Wang, X., Cattaneo, F., Ryno, L., Hulleman, J., Reixach, N., and Buxbaum, J. N. (2014). The systemic amyloid precursor transthyretin (TTR) behaves as a neuronal stress protein regulated by HSF1 in SH-SYSY human neuroblastoma cells and APP23 Alzheimer's disease model mice. J. Neurosci. 34, 7253-7265.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a compound” includes one or more of such compound, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. An isolated cell, wherein said cell is a primate cortical neuronal cell that is BMI1-deficient, and wherein said cell displays one or more phenotypic hallmark of Alzheimer's disease.

2. The cell of claim 1, wherein said one or more phenotypic hallmark is selected from the group consisting of: axonal swelling, axonal segment breaks, beta-amyloid accumulation, C99 fragment accumulation, p-Tau accumulation, synaptic atrophy, neuronal apoptosis, heterochromatin relaxation using H3K9me3 antibody, F-Actin bundles formation using Phalloidin staining, and combinations thereof.

3. The cell of claim 1, wherein BMI1 has been deleted, or wherein BMI1 genetic activity, BMI gene expression, BMI1 protein function, and/or BMI1 protein expression has been reduced and/or inactivated.

4. The cell of claim 1, wherein BMI1 has been inactivated through genetic inactivation using one or more of: a micro-RNA against BMI1 gene, a siRNA against BMI1 gene, a shRNA against BMI1 gene, a genetic inactivation of the BMI1 gene using CRE-Ioxp, a genetic inactivation of the BMI1 gene using CRISPR/Cas9, a genetic inactivation of the BMI1 gene using TALEN, a genetic inactivation of the BMI1 gene using ZFNs, and combinations thereof.

5. The cell of claim 1, wherein said primate is a human.

6. The cell of claim 5, wherein said cell is a human induced pluripotent stem (iPS) cell or a human embryonic stem cell.

7. The cell of claim 1, wherein said Alzheimer's disease is sporadic Alzheimer's disease.

8. A cellular model of a dementia-related neurological disease, comprising an in vitro culture of a plurality of cortical neuronal cells according to claim 1.

9. The cellular model of claim 8, wherein the cell of claim 1 are cultured in three dimensions.

10. The cellular model of claim 8, wherein said cellular model generates amyloid plaques and Tau-tangles.

11. The cellular model of claim 8, wherein said neuronal cell are cultured in a 3D Matrigel™ matrix.

12. The cellular model of claim 8, further comprising a co-culture with astrocytes.

13. The cellular model of claim 12, wherein the cortical neuronal cells and the astrocytes cells are human cells.

14. The cellular model of claim 8, wherein said dementia-related neurological disease is Alzheimer's disease.

15. The cellular model of claim 8, wherein said dementia-related neurological disease is frontotemporal dementia or dementia with Lewy Bodies.

16-17. (canceled)

18. A method for screening anti-Alzheimer drugs, comprising:
exposing the cell of claim 1 to a candidate anti-Alzheimer compound;
assessing said cell for one or more phenotypic hallmark of Alzheimer's disease in presence of said candidate compound; and
selecting a candidate compound capable of inhibiting and/or reducing said one or one or more phenotypic hallmark of Alzheimer's disease.

19-29. (canceled)

30. A method for designing anti-Alzheimer drugs, comprising the steps of:
(a) exposing the cell of claim 1 to a candidate anti-Alzheimer compound;
(b) assessing said cell for one or more phenotypic hallmark of Alzheimer's disease;
(c) selecting a candidate compound capable of inhibiting and/or reducing said one or one or more phenotypic hallmark of Alzheimer's disease;
(d) modifying the chemical structure of candidate compound of step (c) to obtain a modified compound with improved anti-Alzheimer activity.

31. The method of claim 30, further comprising repeating steps (a) to (c) with said modified compound, and optionally repeating step (d).

32. A method for identifying a potential biological target of an anti-Alzheimer drug, comprising the steps of:
(a) making a quantitative proteomic comparative analysis or a genome wide expression comparative analysis of the cell of claim 1 with wild-type cortical neurons;
(b) identifying genes or proteins for which expression or post-translational modification is different;
wherein a gene or protein identified at step (b) is a potential biological target of an anti-Alzheimer drug.

33. The method of claim 32, further comprising the steps of:
(c) exposing the cell of claim 1 and/or the wild-type cortical neurons to an anti-Alzheimer drug;
(d) making a comparative quantitative proteomic analysis or comparative gene expression analysis of the exposed cell and/or cortical neurons with the quantitative proteomic analysis of step (a).

34-42. (canceled)
